(19) United States
(12) Patent Application Publication
(10) Pub. No.: US 2024/0398915 A1
(43) Pub. Date: Dec. 5, 2024

(54) NEMATODE VACCINE

(71) Applicant: AGRESEARCH LIMITED, Hamilton (NZ)

(72) Inventors: Saleh UMAIR, Palmerston North (NZ); Jacqueline Sarah KNIGHT, Palmerston North (NZ)

(21) Appl. No.: 18/697,123

(22) PCT Filed: Oct. 3, 2022
(86) PCT No.: PCT/IB2022/059394

§ 371 Date: Mar. 29, 2024

(30) Foreign Application Priority Data
Oct. 4, 2021 (NZ) 780917

(51) Int. Cl. A61K 39/00; A61K 39/385; A61P 33/10

(52) U.S. Cl. CPC A61K 39/0003; A61K 39/385; A61P 33/10; A61K 2039/552; A61K 2039/55505; A61K 2039/5555; A61K 2039/55566; A61K 2039/55572; A61K 2039/55594; A61K 2039/575

(57) ABSTRACT

The present invention is directed to a vaccine comprising recombinant antigens derived from the parasitic nematode Haemonchus contortus, which will raise an immune response in farmed and wild ruminants that are susceptible or predisposed to infection by one or more nematode worm species. The recombinant antigens used in the invention are conserved among species of nematode worms so that the vaccine will provide protection against multiple types of nematode worms. In particular, the invention provides a composition or vaccine composition comprising the recombinant H. contortus antigens: (i) enolase (EN); (ii) arginine kinase (AK); and (iii) ornithine decarboxylase (ODC), or antigenic fragments thereof, together with a veterinary acceptable carrier or diluent.

### FIELD OF THE INVENTION

The present invention relates to a vaccine comprising antigens which stimulate or boost acquired immunity against infection by parasitic nematodes, particularly in farmed or wild ruminants such as sheep, cattle, goats, deer, buffalo, bison, camelids, llamas, etc.

### BACKGROUND OF THE INVENTION

Parasitic nematode worm infection is one of the biggest health problems for farmed ruminants worldwide. Parasitic worm infections are harmful to a host animal for many reasons. For example, they deprive the host of food, damage internal tissues and organs, cause anaemia, weight loss, diarrhoea, dehydration and loss of appetite. Such parasitic infections cause costly production losses and if left untreated, animals can die causing further economic loss to farmers.

Currently, farmers rely on the use of anthelmintic agents (such as benzimidazoles, levamisole, morantel, monepantel, oxfendazole or ivermectin) to control parasitic nematodes, however resistance of parasites to one or more of these agents is now widespread. Indeed, recent industry-funded surveys in New Zealand found that 64% of sheep farms and 94% of beef farms now have parasites that are resistant to at least one of these anthelmintics.

Such resistance in terms of control and productivity losses is estimated to cost the New Zealand livestock industry around $700 million annually.

Alternative methods of controlling the effect of on-farm parasite infections have been proposed and includes altered grazing management, use of nematode trapping fungi, dietary supplements, selective breeding of animals for host resistance and vaccines.

Attempts to develop recombinant vaccines against parasitic nematodes have met with limited success and to date there are no commercial recombinant vaccines available for any nematode parasites. However, the development of such a vaccine is viewed by the industry as a solution to the resistance problem.

One target for a protective vaccine is against essential worm metabolic enzymes. Parasitic nematode larvae grow rapidly and adult worms lay large numbers of eggs, both requiring highly active nitrogen and energy metabolism. Essential worm enzymes involved in these pathways, and which are not present in the host, are therefore potential targets for controlling parasites. Essential enzymes involved in blood digestion and other pathways critical to the life cycle of the worm could also be targeted either alone or as multiple targets.

Vaccination with antigens comprising such metabolic enzymes would in theory generate circulating host antibodies which would bind to and disrupt the function of the essential parasitic metabolic enzymes, hopefully leading to a substantially reduced worm burden and faecal egg count (FEC).

A vaccine (Barbervax) based on an extract of adult H. contortus has recently been released commercially in Australia. While the vaccine is effective in protecting sheep against infection, there are a number of real or potential issues with its use. First, the vaccine does not provide any long-term protection against infection, and as a result needs to be applied on several occasions over the period of risk. Second, there is significant risk of degradation of the native antigen should it be subjected to high temperatures in the field. Third, as the antigen is extracted from worms derived from donor sheep, there may be a risk of cross-contamination with infectious agents such as viruses.

Recombinant antigens would overcome these issues, however, attempts to make commercial vaccines from recombinant antigens have so far failed. There is therefore a need in the art to provide such recombinant vaccines.

It is an object of the present invention to go some way towards overcoming this need and/or to provide the public with a useful choice.

### SUMMARY OF THE INVENTION

The present invention is directed to a vaccine comprising recombinant antigens derived from the parasitic nematode Haemonchus contortus, which will raise an immune response in farmed and wild ruminants that are susceptible or predisposed to infection by one or more nematode worm species. The recombinant antigens used in the invention are conserved among species of nematode worms so that the vaccine will provide protection against multiple types of nematode worms.

In a first embodiment, the invention provides a composition or vaccine composition comprising the recombinant H. contortus antigens:
(i) enolase (EN);
(ii) arginine kinase (AK); and
(iii) ornithine decarboxylase (ODC), or antigenic fragments thereof, together with a veterinary acceptable carrier or diluent.

The composition or vaccine composition may further comprise one or more recombinant H. contortus antigens selected from the group consisting of:
(iv) seryl tRNA synthetase (SRS-2);
(v) macrophage migration inhibitory factor 2 (MIF-2);
(vi) fatty acid synthetase (FASN-1);
(vii) NAD (P) H-dependant oxidoreductase (F36A2-3);
(viii) glutamyl tRNA synthetase (ERS-2);
(ix) aspartyl tRNA synthetase (DRS-1);
(x) transcriptional co-activator (CBP-1);
(xi) vacuolar ATPase (VHA-12); and
(xii) serum-glucocorticoid-inducible kinase (SGK-1), or antigenic fragments thereof.

Preferably, the composition or vaccine composition comprises at least one, at least two, at least three, at least four, at least five, at least six, at least seven, at least eight, or at least nine, of the antigens (iv) to (xii), above.

In a second embodiment, the invention provides a composition or vaccine composition comprising the H. contortus recombinant antigens:
(i) enolase (EN);
(ii) arginine kinase (AK);
(iii) ornithine decarboxylase (ODC);
(iv) seryl tRNA synthetase (SRS-2);
(v) macrophage migration inhibitory factor 2 (MIF-2);
(vi) fatty acid synthetase (FASN-1);
(vii) NAD (P) H-dependant oxidoreductase (F36A2-3);
(viii) glutamyl tRNA synthetase (ERS-2);
(ix) aspartyl tRNA synthetase (DRS-1);
(x) transcriptional co-activator (CBP-1);
(xi) vacuolar ATPase (VHA-12); and
(xii) serum-glucocorticoid-inducible kinase (SGK-1),
   or antigenic fragments thereof, together with a veterinary acceptable carrier or diluent.

The composition or vaccine composition may further comprise an adjuvant, such as: alum, Quil A, Freund's complete adjuvant, Freund's incomplete adjuvant, lipopolysacharride, monophosphoryl lipid A, montanide, lipovant, bacterial flagellin, adjuvant 65, gamma inulin, algammulin, imiquimod, guardiquimod, murimyl dipeptide, etc.

The composition or vaccine composition may further comprise a carrier such as: a chitin-based slow release compound (sol-gel), hollow mesoporous silicon nanoparticles (HMSNs), poly(d,l-lactide-co-glycolide) (PGC) nanoparticles, poly(d,l-lactic-coglycolic acid) (PGCA) nanoparticles, liposomes, virosomes, cochleate delivery vehicles, etc.

In a third embodiment, the invention provides a method of reducing parasitic nematode worm burden in a farmed or wild ruminant animal, said method comprising administering an effective amount of the composition or vaccine composition of the invention to said ruminant animal on one or more occasions, whereby parasitic worm burden reduction is measured by a reduced faecal egg count (FEC), and/or an increase in expulsion of larvae and/or adult nematode worms.

In a fourth embodiment, the invention provides a method of inducing an immune response in a farmed or wild ruminant animal to treat or protect said animal against infection by parasitic nematodes, said method comprising administering an effective amount of the composition or vaccine composition of the invention to said animal on one or more occasions, wherein induction of an immune response is measured by the presence of protective antibodies against one or more specific antigens present in said composition or vaccine composition.

In a fifth embodiment, the invention provides a method of stimulating or boosting acquired immunity in a farmed or wild ruminant animal to treat or protect said animal against infection by parasitic nematodes, said method comprising administering an effective amount of the composition or vaccine composition of the invention to said animal on one or more occasions, wherein stimulation or a boost of said acquired immunity is measured by one or more of: the presence of protective antibodies against one or more specific antigens present in said composition or vaccine composition; an increased level of cytokines; a reduced FEC; and/or expulsion of larvae and/or adult nematodes.

In a sixth embodiment, the invention provides a method of treating or preventing a nematode infection in a farmed or wild ruminant animal comprising administering an effective amount of said composition or vaccine composition to said animal.

In a seventh embodiment, the invention provides a use of the recombinant H. contortus antigens (i) enolase (EN), (ii) arginine kinase (AK), and (iii) ornithine decarboxylase (ODC), or antigenic fragments thereof, in the manufacture of a composition or vaccine composition for reducing nematode parasitic worm burden in a farmed or wild ruminant animal.

In an eighth embodiment, the invention provides a use of the recombinant H. contortus antigens (i) enolase (EN), (ii) arginine kinase (AK), and (iii) ornithine decarboxylase (ODC), or antigenic fragments thereof in the manufacture of a composition or vaccine composition for stimulating or boosting acquired immunity in a farmed or wild ruminant animal to treat or protect said animal against infection by parasitic nematodes.

In a ninth embodiment, the invention provides a use of the recombinant H. contortus antigens (i) enolase (EN), (ii) arginine kinase (AK), and (iii) ornithine decarboxylase (ODC), or antigenic fragments thereof, in the manufacture of a composition or vaccine composition for treating or preventing a nematode infection in a farmed or wild ruminant animal.

The composition or vaccine composition used in these embodiments of the invention may further comprise one or more of antigens (iv)-(xii), above.

The farmed or wild ruminant animal is selected from the group consisting of sheep, cattle, goat, deer, buffalo, bison, camelids, llamas etc. The farmed or wild ruminant animals are preferably young animals, less than one year old, i.e. lambs, calves, kid goats etc. In one aspect, the farmed or wild ruminant animal is less than 6 months old. In a further aspect, the farmed or wild ruminant animal is at least 3 months old.

The parasitic nematodes treatable by the methods of this invention include Trichostrongylus colubriformis, Haemonchus contortus, Haemonchus placei, Ostertagia (Teladorsagia) circumcincta, Cooperia curticei, Nematodirus spathiger, Trichostrongylus axi, Trichostrongylus vitrinus, Ostertagia ostertagia, Cooperia oncophera, Nematodirus brasiliensis, Dictyocaulus eckerti, Strongylus vulgaris, Toxascaris vitolorum, Nematodirus filicollis, Ashworthius sidemi, Mecistocirrus digitatus, Bunostomum trigonocephalum, Trichuris discolor, Toxacara vitulorum, etc.

### BRIEF DESCRIPTION OF THE DRAWINGS

The invention will now be described in more detail with reference to the accompanying drawings in which:

FIGS. 1a-c: shows the degree of homology of the metabolic enzymes AK (FIG. 1a), EN (FIG. 1b) and ODC (FIG. 1c) across nematode species as follows:

FIG. 1a shows comparison of predicted arginine kinase amino acid sequences from the members of the Strongylida: Haemonchus contortus (Genebank Accession No. AFT82971), Teladorsagia circumcincta (AFT82970), Necator americanius (ETN81593) and Ancylostoma ceylanicum (EYC23758; EYC23757; EYB91576), and from members of the Rhabditidae: Caenorhabditis elegans (CAB00062; NP509217; NP507054; CCD73398; CCD79398), Caenorhabditis briggsae (CAP24981; CAP24932), Caenorhabditis brenneri (EGT52941; EGT41918) and Caenorhabditis remanei (EFP12066; EFO86450; EFO82749);

FIG. 1b shows comparison of predicted enolase amino acid sequences from members of the Strongylida: Haemonchus contortus (Genebank Accession No. AGC24386; ADK47524; CDJ96217), Teladorsagia circumcincta (deduced from T. circumcincta genome sequence), Mecistocirrus digitatus (BAN67669), Ancylostoma ceylanicum (EYB81234), Angiostrongylus cantonensis (AGO81688), Necator americanus (ETN80540), and from members of the Rhabditidae: Caenorhabditis elegans (NP495900; NP871916; NP001022349), Caenorhabditis brenneri (EGT35078) Caenorhabditis briggsae (CAP23453), and Caenorhabditis remanei (EFO85696);

FIG. 1c shows comparison of predicted ornithine decarboxylase amino acid sequences from members of the Strongylida: Haemonchus contortus (Genebank Accession No. AAC27893), Teladorsagia circumcincta (AGH70348) and Ancylostoma ceylanicum (EYC11973; EYC11971; EYC11970), and from members of the Rhabditidae: Caenorhabditis elegans (P41931), Caenorhabditis briggsae (CAP36352), Caenorhabditis brenneri (EGT47038) and Caenorhabditis remanei (EFP05480).

The alignments in FIGS. 1a-c were performed using the Muscle alignment option in Geneious 5.6.5 (Biomatters Ltd) with the Blosum 62 similarity matrix used to determine 100% similar residues (shaded). The consensus sequence shown is for the most common residue with the fewest ambiguities;

FIG. 2: shows antibody response against recombinant AK, EN and ODC in sheep when Quil A was used as adjuvant;

FIG. 3: shows antibody response against recombinant AK, EN and ODC in sheep when alum was used as adjuvant;

FIG. 4: shows the eggs per gram (EPG) in response to the Quil A and alum vaccines of a first sheep trial;

FIG. 5: shows the EPG of a second sheep trial in response to a vaccine comprising 3 antigens (recombinant AK, EN and ODC 3AgV) or 12 antigens (recombinant AK, EN, ODC, SRS-2, MIF-2, FASN-1, F36A2-3, ERS-2, DRS-1, SGK-1, VHA-12 and CBP-1 (12AgV)).

FIG. 6: shows the worm burden of sheep treated with 3AgV and 12AgV compared to controls;

FIG. 7: shows the haematocrit values of sheep from 5 to 8 weeks after vaccination with 3AgV or 12AgV compared to controls;

FIG. 8: shows the live weight of sheep in response to vaccination with 3AgV or 12AgV compared to control;

FIG. 9: shows the antibody response to each antigen in pooled serum of sheep vaccinated with 3AgV at day 0 and again at day 34;

FIG. 10: shows the antibody response to each antigen in pooled serum of sheep vaccinated with 12AgV at day 0 and again at day 34;

FIG. 11: shows faecal egg counts of sheep in the third trial for 3AgV, 7AgV and 11AgV and control groups;

FIG. 12: shows male, female and total adult worm counts in vaccine and control groups. Data was log transformed for analysis. 3AgV, 7AgV and 11AgV were significantly different from PosCt and AdjCt.

FIG. 13: shows mean weight gains of treatment and control groups through the course of the trial. X-axis represent days post-first vaccination whereas the Y-axis represent weight in kgs. There were no statistical differences amongst any groups;

FIG. 14: shows that vaccination reduces the decline of haematocrit levels during challenge with H. contortus;

FIG. 15: shows the EPG of a calf trial in response to a vaccine comprising 3AgV and 11AgV, compared to control calf group;

FIG. 16: shows total adult worm count in vaccine and control groups. Data were log-transformed for analysis. 3AgVac and 11AgVac were significantly different from control calf group.

FIG. 17: shows vaccination reduced the decline of haematocrit levels during challenge with H. contortus in 3AgV and 11AgV treated calves compared to control calves:

FIG. 18: shows antibody responses (IgG) in serum samples against 11AgV. Antibody levels of the vaccine (solid line) and mean of control (dotted line) groups animals from week 1 to week 12. Antibody levels were measured by ELISA on sera diluted 1:4000 at optical density 450 nm; and

FIG. 19: shows antibody responses (IgG) in goat kid serum samples against 11AgV. Antibody levels were measured by ELISA on sera diluted 1:4000 at an optical density of 450 nm. Antibody levels of the vaccine (dotted line) and control (solid line) groups animals from week 1 to week 14. Arrow heads indicate the week of vaccination.

### DETAILED DESCRIPTION

Nematode worm infestation of farmed and wild ruminants is a major problem around the world and Haemonchus contortus is the most damaging of these nematode worms.

The only successful protein-based vaccine against H. contortus to date was made from isolated native proteins. All attempts to date to make a vaccine from recombinant proteins have failed.

The present invention provides for the first time an effective vaccine against nematode worm infestation in farmed and wild ruminants comprising a mixture of recombinant antigens.

The recombinant antigens correspond to H. contortus metabolic enzymes that are obligatory for worm survival.

The term “antigen” used herein means a molecule that provokes an immune response involving antibody production.

Without wishing to be bound by theory it is thought that antibodies produced as a result of immunisation with the vaccine composition of the invention act in two main ways. Firstly, for blood sucking nematodes, the antibodies will be ingested by the parasitic nematode worms in the host blood during feeding. Ingested antibodies will bind to target antigens, in this case, essential metabolic enzymes present in the intestinal wall of the nematode or secreted into the intestine cavity, thereby inhibiting their activity resulting in weakness of the worms which are then removed from the gut of the host animal by peristalsis. For non-blood sucking nematodes, the antibodies generated by the vaccine composition of the invention, include antibodies directed against antigens found in worm somatic tissue and/or secretory/excretory products affecting the worms ability to survive in the host intestine. The worms become weak and are expelled.

Efficacy of the vaccine composition of the invention can be measured by an increase in expulsion of larvae and/or adult nematodes, and/or by a reduced faecal egg count (FEC), as well as by the presence of one or more protective antibodies targeted by the antigens present in the vaccine composition.

The antigens present in the composition or vaccine composition of the present invention comprise (i) recombinant H. contortus enolase (EN), (ii) recombinant H. contortus arginine kinase (AK), and (iii) recombinant H. contortus ornithine decarboxylase (ODC), or antigenic fragments thereof.

Enolase is an enzyme involved in the glycolytic pathway and is a secreted enzyme forming part of the excretory/secretory (ES) complex. Enolase plays a vital role in the metabolism of nematode worms (Han et al, 2012). Arginine kinase is thought to be present in the cells lining the parasite gut and plays a vital role in the maintenance of ATP levels. Inhibition of these enzymes by antibodies raised in response to inoculation of the vaccine of the present invention is shown for the first time to result in a significant reduction in faecal egg count (FEC), worm burden and other symptoms of H. contortus infestation in sheep.

Enolase and arginine kinase are highly conserved enzymes across nematode worm species so that the vaccine of the present invention is anticipated to be effective against a host of nematodes that infect farmed and wild ruminants including Bunostomum, Strongylus, Trichostrongylus, Haemonchus, Ostertagia, Toxascaris, Nematodirus, Trichuris, Dictyocaulus, Toxocara, Strongyloides, Cooperia, Ashworthius and Mecistrocirrus.

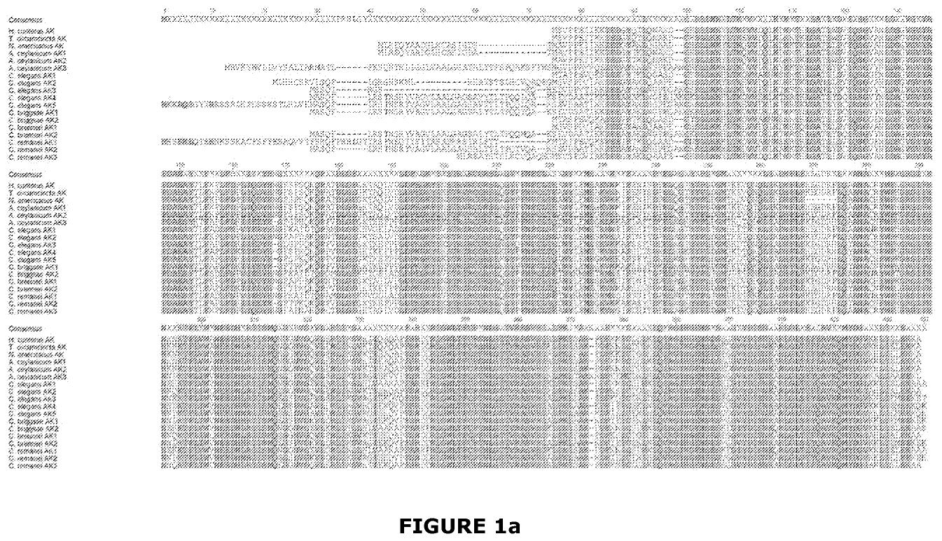

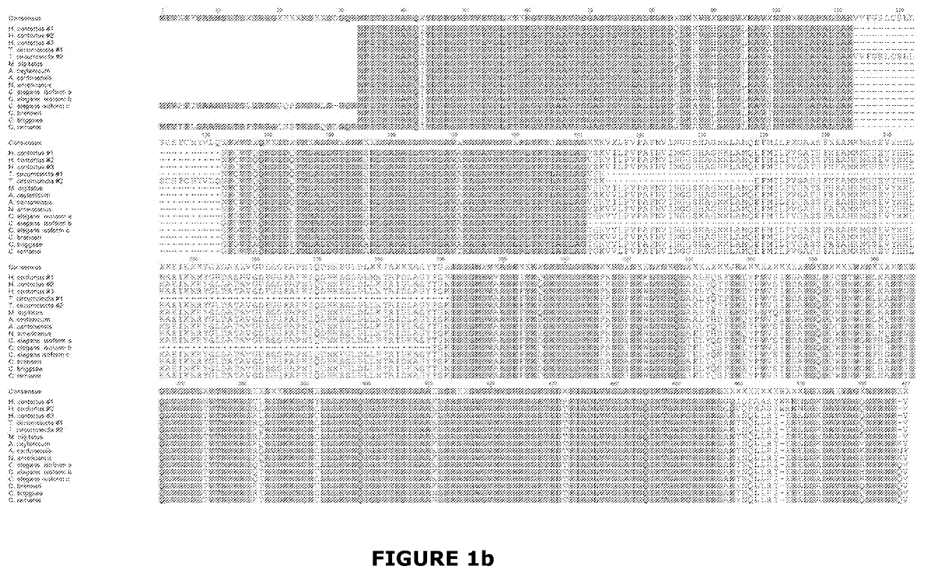

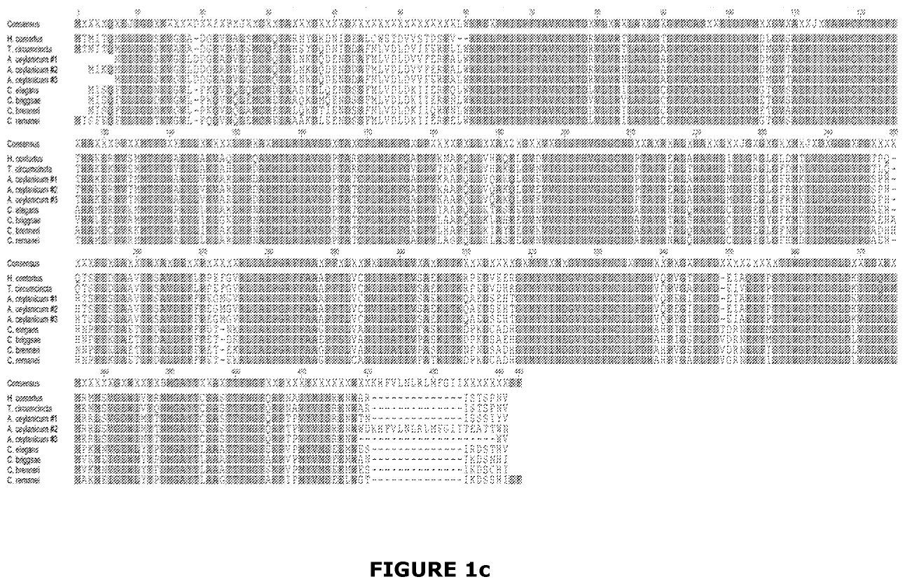

The degree of homology of EN and AK across nematode worm species is shown in FIGS. 1a and 1b, and in Table 1, below.

As will be understood by a skilled worker, it is expected that, as there is such high conservation of EN and AK across species, the vaccine containing these H. contortus antigens will raise antibodies that will recognise EN and AK of other nematode species and work in the same way to block enzyme activity and so impact detrimentally on worm survival.

TABLE 1
The % of identical amino acid residues shared with Haemonchus contortus
arginine kinase (H. contortus AK; GenBank Accession No. AFT82871), Haemonchus
contortus enolase (H. contortus #1; AGC24386) and Haemonchus contortus ornithine
decarboxylase (H. contortus; AAC27893).

| arginine kinase | | enolase | | ornithine decarboxylase | |
|---|---|---|---|---|---|
| | % | | % | | % |
| Species | Identity | Species | Identity | Species | Identity |
| H. contorus AK | | H. contortus #1 | | H. contortus | |
| T. circumcincta AK | 100 | H. contortus #2 | 94.7 | T. circumcincta | 86.6 |
| N. americanus AK | 94.2 | H. contortus #3 | 91.9 | A. ceylanicum #1 | 77.3 |
| A. ceylanicum AK1 | 96.9 | T. circumcincta #1 | 69.8 | A. ceylanicum #2 | 73.9 |
| A. ceylanicum AK2 | 96.9 | T. circumcincta #2 | 80.8 | A. ceylanicum #3 | 76.6 |
| A. ceylanicum AK3 | 68.8 | M. digitatus | 87.1 | C. elegans | 62.4 |
| C. elegans AK1 | 72.4 | A. ceylanicum | 88 | C. briggsae | 63.2 |
| C. elegans AK2 | 87.5 | A. cantonensis | 86.9 | C. brenneri | 62.5 |
| C. elegans AK3 | 72 | N. americanus | 87.6 | C. remanei | 62.4 |
| C. elegans AK4 | 71.7 | C. elegans isoform a | 83 | | |
| C. elegans AK5 | 72 | C. elegans isoform b | 63.9 | | |
| C. briggsae AK1 | 71.5 | C. elegans isoform c | 83 | | |
| C. briggsae AK2 | 72.4 | C. brenneri | 84.1 | | |
| C. brenneri AK1 | 88 | C. briggsae | 83.7 | | |
| C. brenneri AK2 | 71.5 | | | | |
| C. remanei AK1 | 71.9 | | | | |
| C. remanei AK2 | 72 | | | | |
| C. remanei AK3 | 88.9 | | | | |

Examples of specific nematode worm species that the vaccine of the present invention can be used to target include Trichostrongylus colubriformis, Haemonchus contortus, Haemonchus placei, Ostertagia (Teladorsagia) circumcincta, Cooperia curticei, Nematodirus spathiger, Trichostrongylus axei, Trichostrongylus vitrinus, Ostertagia ostertagia, Cooperia oncophera, Nematodirus brasiliensis, Dictyocaulus viviparus, Dictyocaulus eckerti, Strongylus vulgaris, Taxascaris vitulorum, Nematodirus filicollis, Ashworthius sidemi, Mecistocirrus digitatus, Bunostomum trigonocephalum, Trichuris discolor, Toxacara vitulorum, etc.

Preferably the parasitic nematode is Haemonchus contortus or Haemonchus placei.

In addition to (i) EN, (ii) AK, and (iii) ODC, the composition or vaccine composition of the present invention can further comprise one or more recombinant H. contortus antigens selected from the group consisting of:
(iv) seryl tRNA synthetase (SRS-2);
(v) macrophage migration inhibitory factor 2 (MIF-2);
(vi) fatty acid synthetase (FASN-1);
(vii) NAD (P) H-dependant oxidoreductase (F36A2-3);
(viii) glutamyl tRNA synthetase (ERS-2);
(ix) aspartyl tRNA synthetase (DRS-1);
(x) transcriptional co-activator (CBP-1);
(xi) vacuolar ATPase (VHA-12); and
(xii) serum-glucocorticoid-inducible kinase (SGK-1), or antigenic fragments thereof.

Preferably, the composition or vaccine composition of the invention further comprises at least one, at least two, at least three, at least four, at least five, at least six, at least seven, at least eight, or all nine, of the antigens (iv)-(xii), above.

The % sequence identity for (iii) ODC across species of nematode worms is shown in FIGS. 1c and 1n Table 1, above. Homology for the remaining antigens (iv)-(xii) is not shown.

It is expected that a composition or vaccine composition of the invention having additional antigens to (i) EN (ii) AK and (iii) ODC, above, will result in a stronger immunogenic response and improved reduction in FEC and worm burden due to at least an additive effect of each individual antigen.

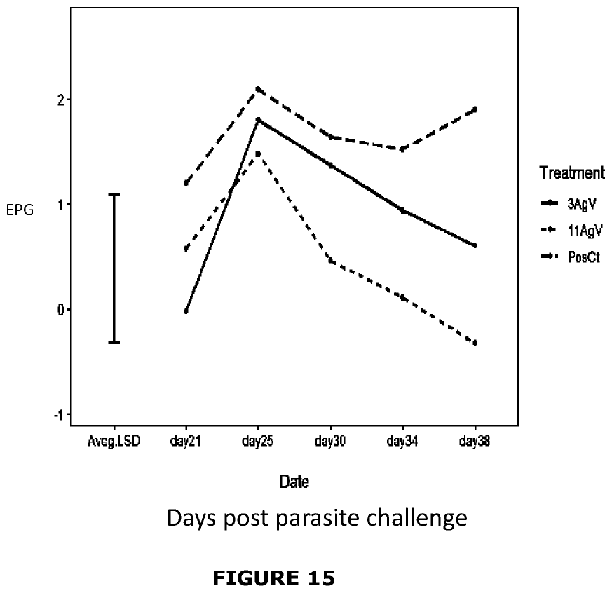

Indeed, the FEC was reduced by 80% using a vaccine composition comprising all twelve antigens compared to about 50% using a vaccine composition comprising three antigens in sheep (see FIG. 5), and by 100% using a vaccine comprising eleven antigens compared to about 50% using a vaccine comprising three antigens in calves (see FIG. 15), discussed below. However, a 50% reduction of FEC is still considered to be a significant reduction and proves efficacy of the 3AgV vaccine composition of the invention.

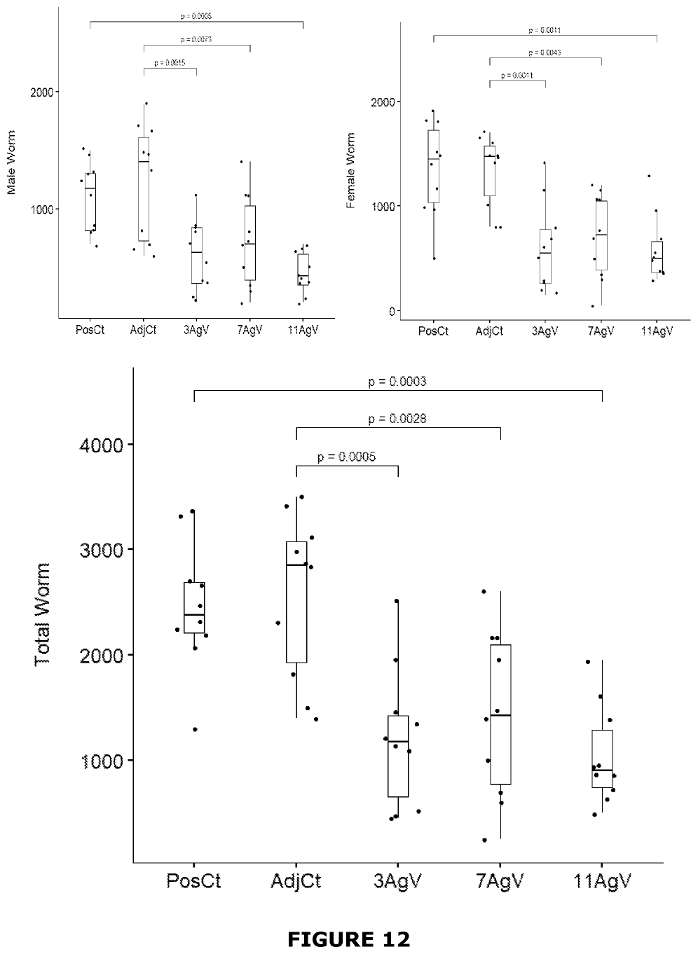

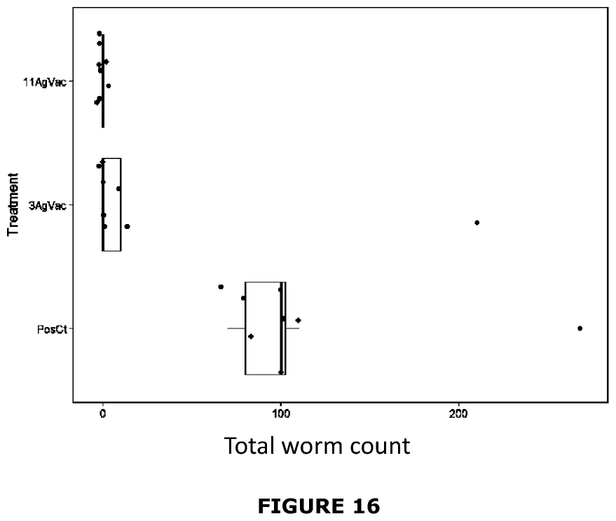

Worm burden was also reduced with increasing antigens in the vaccine compositions of the present invention. Total adult worms as well as total male and female worms were significantly reduced as a result of vaccination in sheep with three, seven and eleven antigens as compared to the control groups. As expected, the eleven antigen vaccine group showed the greatest reduction in the adult worms compared with control groups and the overall adult worm reduction was just over 60% (see FIG. 12). This finding was repeated in calves where an eleven antigen vaccine surprising reduced worms in calves by 100% compared to 75% using a vaccine comprising three antigens (FIG. 16). The reduction in adult worm count with the vaccine compositions of the invention was significant and sufficient to prove efficacy of a vaccine composition comprising from three to eleven antigens.

It is also envisaged that the vaccine composition of the present invention will also be effective using antigenic fragments of H. contortus EN, AK and ODC as would be understood by a skilled worker.

Antigenic fragments of the optional recombinant antigens (iv)-(xii), above, may also be used in the vaccine composition of the invention.

An antigenic fragment is understood to mean a fragment of any one or more of antigens (i)-(xii) that will have effective antigenic properties, i.e. will result in the generation of antibodies that will recognise and bind to the corresponding worm proteins. A skilled worker is easily able to test fragments of antigens (i)-(xii) to determine antigenicity, i.e. antibody response, by performing enzyme-linked immunosorbent assay (ELISA) against immune or naive sheep saliva and serum using standard procedures.

Alternatively, species-specific recombinant homologs of the H. contortus antigens, or fragments of recombinant homologs of the H. contortus antigens, can be used that have at least 70%, at least 75%, at least 80%, at least 85%, at least 90%, at least 95%, at least 98%, or at least 99% sequence identity thereto as would be understood by a skilled worker. Such recombinant homologs can be identified and produced using known technology.

In addition, corresponding native antigens can be used in place of or together with the recombinant antigens disclosed herein, as would be understood by a skilled worker, bearing in mind the problems associated with native antigens, as discussed in the background section.

In a further embodiment, the composition or vaccine composition of the invention comprises the recombinant H. contortus antigens (i) EN of SEQ ID NO:1, (ii) AK of SEQ ID NO:2, and (iii) ODC of SEQ ID NO:3; or antigenic fragments thereof, together with a veterinary acceptable carrier or diluents.

The composition or vaccine composition may further comprise one or more of the recombinant H. contortus antigens selected from the group consisting of:
(iv) SRS-2 of SEQ ID NO:4;
(v) MIF-2 of SEQ ID NO:5;
(vi) FASN-1 of SEQ ID NO:6;
(vii) F36A2-3 of SEQ ID NO:7;
(viii) ERS-2 of SEQ ID NO:8;
(ix) DRS-1 of SEQ ID NO:9;
(x) CBP-1 of SEQ ID NO:10;
(xi) VHA-12 of SEQ ID NO:11; and
(xii) SGK-1 of SEQ ID NO: 12, referred to in Table 2, below, or antigenic fragments thereof.

In one embodiment the composition or vaccine composition of the invention may comprises antigens comprising at least 70-99% sequence identity to SEQ ID NOS: 1-12.

TABLE 2
Haemonchus contortus Antigen Protein Sequences

Haemonchus contortus enolase (EN) protein sequence (SEQ ID NO: 1)
MPITKIHARQIYDSRGNPTVEVDLYTEKGVFRAAVPSGASTGVHEALELRDQDKKVHHGKGVLKA
VANINDKIAPALIAKNFCVTQQRDIDQFMLALDGTENKSNLGANAILGVSLAVAKAGAVHKGMPL
YKYIAELAGVSKVILPVPAFNVINGGSHAGNKLAMQEFMILPVGATSFHEAMRMGSEVYHHLKAEI
KKRYGLDATAVGDEGGFAPNIQDNKEGLDLLKTAIDLAGYTGKISIGMDVAASEFYKQGKYDLDF
KNPKSDPSKWLTGDQLAALYQTFIKEYPVVSIEDAFDQDDWDNWGKLKASTNIQLVGDDLTVTN
PKRIRLAIDKKSCNCLLLKVNQIGSVTESIEAAKLSRSNGWGVMVSHRSGETEDTFIADLVVGLAT
GQIKTGAPCRSERLAKYNQLLRIEEELGKDAVYAGQNFRNPV
Haemonchus contortus arginine kinase (AK) protein sequence (SEQ ID NO: 2)
MSVPPEIIKKIEDGYQTLQNAKDCHSLLKKYLTKEVVDQLKDKKTKLGATLWDVIQSGVANLDSG
VGVYAPDAEAYTLFKPLFDPLIQDYHNGFSPSQKQPATDLGEGKTAQLVDLDPEGKYINSTRVRC
GRSLQGYPFNPCLTEANYLEMEAKVKKIFENISDPELQGTYYPLDGMTKEVQNQLIKDHFLFKEGD
RFLQAANACRYWPKGRGIFHNKNKTFLVWANEEDHLRIISMQNGGNVGQVLERLIKGVKIIQAQ
APFSRDDRLGWLTFCPSNLGTTVRASVHIRLPKISAKPDFKKICDDLKLQIRGIHGEHSDSEGGVY
DISNKARLGLTEFEAVKQMYDGVKHLIELEKKA
Haemonchus contortus ornithine decarboxylase (ODC) protein sequence (SEQ
ID NO: 3)
MTMITQMELIGDSKVGIADGEVDALSMCRQIARNYDKDNIDEALCWSTDVVSTDSFVKRELPMI
EPFYAVKCNTDRVLVRTLAALGTGFDCASREEIDIVMDLGVSAERIVYANPCKTRSFITHAKERNV
SMMTFDSAEELAKVAQLHPQAKMILRIAVSDPTARCPLNLKFGADPVKMAPQLLVHAQELGVDVI
GISFHVGSGCNDPTAYREALAHARHLIELGRGLGLDMTLVDLGGGYPGTPQQTSFEDIAAVIRSA
VDEYLPPEFGVRLIAEPGRFFAAAPFTLVCNIIHATEVSAEKITKRPEDVEERGFMYYVNDGVYGSF
NCILFDHVQPVGTPLFDEIAQEYPSTIWGPTCDSLDKIEDQKLMRMMSVGEWIVYRNMGAYTCS
ASTTFNGFQRPNAIYMINRKNWARISTSPNV
Haemonchus contortus seryl tRNA synthetase (SRS-2) protein sequence (SEQ
ID NO: 4)
MVLDMDLFREEKGGNPEAIRNSQRQRYCDPSIVDKVIELDQAWRKERFLLDVLNRQKNVLSKAI
GEKVKKKEAQGTDDNVDDSIVSKLESLKVEDLSALTVVQIKKLRVLLDEKMNETKASMEKLEDD
RHQSLIQIGNIIHHSVPVSDDEANNRVERTHGDITSRKKYSHVDLVVMIDGFDGERGTAVAGGR
GYFLKGPLVFLEQAIIQLALQKLGEKGFTPLYTPFFMRKEVMQEVAQLSQFDGELYKVNSKGSEVL
GDNSIDEKYLIATSEQPIAAFHRNEWIKESDLPIKYAGISTCFRQEVGSHGRDTRGIFRVHQFEKV
EQFVICSPLNNESWKIFDEMINNAEEYCQLLGIPYQIVCIVSGELNNAASKKLDLEAWFPGSGAFR
ELVSCSNCTDYQARRLKVRYGMTKKMDGEVPFVHMLNATMCATTRVLCALLENYQTEDGITVPE
VLHPFMPAKYRTFIPFVKPAPIDEESKKKSGK
Haemonchus contortus macrophage migration inhibitory factor 2 (MIF-2)
protein sequence (SEQ ID NO: 5)
MPMVRVATNLPDKDVPANFEERLTDLLAESMNKPRARIAVEMMAGQRIMHGGVRNPVVLIKVESI
GALDPDSTIRHTQKVTQLCTEVLHVPKDKVIISYFDLAPTNVGFAGTTVAAAT
Haemonchus contortus fatty acid synthetase thioesterase domain (FASN-1)
protein sequence (SEQ ID NO: 6)
TRALQPTELDMKKESERDAEQNTVEMLEKQMNQLFKMRVDVNDLDPQDIVVKCNKIEEGPVTFF
VHSIEGIATPLKRVMTKCTFPVYCFQSTKEVPQDSIESVAKCYIREMKKIQPAGPYRIVGYSYGACI
GFEMATMLQESDGPSAVERLILLDGSHLYMQTYRNVYRMAFGVTGDTLVNNPLFESEIMCAMTLR
FANVDYKKFRVELLQQPGFKARIQKVVDTVMTTGLFKSPETIAFACEAMRSKFLMADKYKPERKFT
GLITLVRAEQGAAREEDVGTDYGISQVADDSKVYVVEGDHDTFVQGKSSAKTVAIINELIKETYKC
Haemonchus contortus NAD(P)H-dependant oxidoreductase (F36A2-3)
protein sequence (SEQ ID NO: 7)
MIIKKQFDESEEIVVSKKELRSFVLNCLEKVSCSPGHAQQLADILICSDYRGHYSHGLNRLHIYVN
DLAEKSTLSDGEPLIIKQKGATAWVDGCNLLGPVVGNFCMKLAIQKARTHGIGWVVAKNSNHFG
IAGWYAESALQQGLVGMAFTNTSPCVFPTNSAEKSLGSNPICLAAPAANGDSFFLDMASTTVAYG
KIEVVDRRGGKRIPRAWGADADGIETQDPKEVLNGGGLQPLGGSEATGGYKGTGLCMMVEILC
GIMAGSSFGKSIRKWQTTDESANLGQCFVAIDPECFAPGFGERLSCFLDETRDLKPVDPSYPVQV
AGDPERAHMYMCDELDGIVYKKSQLEHLNKLANDLGVHMFKAKKMTSSLETDGQSRIELEV
Haemonchus contortus glutamyl tRNA synthetase (ERS-2) protein sequence
(SEQ ID NO: 8)
MVAVKQQLVINAKQKEVPYAAALAIAFGGYNPCLSIAFNEKEPVGMNMDGSLIRNDVAIARIVAQ
SLGLPEFTGSTCFEIAKIDEVLTLCEKLQEKFSALFNAIVEDQRFVAAHMMVGKFTTAKIAKPLSKE
KQRDEGKFVELPGAEKGKVVVRFPPEASGYLHIGHAKAALLNQYYQQTFEGQLIMRFDDTNPAKE
NAHFEKVIKEDLAMLNIIPDRWTHSSDYFELMLQMCEKLLREGKAYVDDTDTETMRKEREERVES
KNRSLSPEANLVLWEEMKKGTERGLQCCVRIKIDMQSNNGAMRDPTIYRCKPEEHVRTGMKYKV
YPTYDFACPIVDSIEGVTHALRTTEYTDRDDQYYFICDALGLRKPFIWSYARLNMTNTVMSKRKLT
WFVNEGLVEGWDDPRFPTVRGVMRRGMTVEGLRQFIIAQGGSRSVVMMEWDKIWSFNKKVID
PVAPRYTALETTAIVPVFISTPVVVQDAEVPLHPKNADVGKKTIWHSAKLLVEQVDAQEMKSGDT
VTFVNWGNIKIVSVNKKNGTVSEIHAVLDLANQDYKKTMKVTWIAEADIPSAACIPVVAIEYDHII
SKAVVGKEEDWKNFINYESVHYTKMLGEPALRSVRKGDIIQLQRKGFYICDHDYQPKSEFSGAES
PLLLIYIPDGHVKEPVNKPKPSSVVAASTGKPGDALDLYKLIEAQGNTVRDLKSKDPKAESTKMAV
QKLLELKKQYSEVTGQEYKPGKVPEPSNKVAASTTNESLALYMKIEAQGELVRTEKAKDAKSEAS
KAAIATLLELKKEYKEKTGQEYKAGQPPATTAPSVGTPPGAITEPSTIYAEIEAQGELVRKEKAKDP
KSETAKAAIAKLLDLKKQYKEQTGQDYKPGQQATSLKSPSLGSGGDAISLYSEIEAQGNLVRQEK
AKDAKSEAAKAAIAKLLELKKKYEEVTGHPYKPGQPPAETPSSPQKTAFDESALYEEIKAQGDLVR
QEKQKDAKSDASKAAIQKLLDLKKLYKEKTGQEYKPK
Haemonchus contortus aspartyl tRNA synthetase (DRS-1) protein sequence
(SEQ ID NO: 9)
MNDAAEGGEKKLSKKELNKLAKAAKIAELKAQKAASQPKEDEGEDVSVGMYGSYGMIQSADKK
DIVFTKLNKIEPDLDGQEVWVRGRVHAIRSKGKTCFLVLRQQFYTAQVTLFVGEKISKQMLKFVS
NISKESIVDIQGLVGKVDVQIESCTQKNAELHAIQVFVVSAAEPRLPLQIEDASRRADNTDGLAAV
NLDTRLDNRVLDLRTTTTQGIFSLQAGVCKLFRDTLTERGFVEIHTPKIISAASEGGANVFTVSYFK
GSAYLAQSPQLYKQMAIAGDFGKVFTIGGVFRAEDSNTHRHMTEFVGLDLEMAFNFHYHEVLETI
GSVLISIFKGLKKDYAAEIEAVGRQYPAEPFEFCEPALVLKYPDAVKMLREDGVEMGDEDDLSTPV
EKQLGRLVKEKYKTDFFILDKFPLAVRPFYTMPDPHDPRYSNSYDMFMRGEEILSGAQRIHDAEFL
VERAKHHNIELEKIQAYIDSFKYGCPPHAGGGIGLERVTMLFLGLHNIRLASMFPRDPKRITP
Haemonchus contortus transcriptional co-activator histone acetyltransferase
domain (CBP-1) protein sequence (SEQ ID NO: 10)
ERYTYCLKCFDASPPEGISLSENPNDQSNMAPKDKFVQMKNNVIDYEPFEVCKYCHRKWHRICAL
YDKKVFPEGFICDTCRKEKNYPKPKNRFMAKRLPHNKLSQFLEDRVNTFLKKALSNSPEQYEVIIR
TLCVQDKEVEVKPLMKSKYGPQGFPDRFNYRTKAVFAFEIIDGVEVCFFGLHVQEYGSNCKEPNA
RRVYIAYLDSVHFFQPRELRTDVYHEILLGYLDYVKRLGYTMAHIWACPPSEGDDYIFHCHPPEQK
IPKPKRLQDWYKKMLEKGVTEKTVVEFKDIYKQARDDNLTTPMSLPYFEGDFWPNVIEDCIREAG
NEEAQRRKEVAEADEEDDDIFQSGDNGKKKSSKNKKNNLKKNSKMNKKKQGNSTGNEVADKL
YSQFEKHKEVFFTIRLVTQQSALSLPDIVDPDPLMASDMMDGRDTFLTRARDEHWEFSSLRRAK
Haemonchus contortus vacuolar ATPase, B subunit (VHA-12) (SEQ ID NO: 11)
MAAVDVNKGITSHKTATIRNYNTQPRLIYKTVTGVNGPLVILNDVKFPQFNEIVHITLPDGSKRSG
QVLEITRNKAVVQVFEGTSGIDAKNTICEFTGDILRSPVSEDMLGRIFNGSGKPIDKGPPVLAEDF
LDINGQPINPWSRIYPEEMIQTGISAIDVMNSIARGQKIPIFSAAGLPHNEIAAQIVRQGGLVQLPE
RKHDASDSNFAIVFAAMGVNMETARFFKQDFEENGSMENVCLFLNLANDPTIERIITPRLALTAAE
FFAYQCEKHVLVVLTDMSSYAEALREVSAAREEVPGRRGFPGYMYTDLATIYERAGRVEGRDGSI
TQIPILTMPNDDITHPIPDLTGYITEGQIYVDRQLHNRQIYPPINVLPSLSRLMKSAIGEGMTREDH
SDVSNQLYACYAIGKDVQAMKAVVGEEALSSDDLLYLEFLVKFEKNFITQGNYENRTVFESLDIG
WQLLRIFPREMLKRIPESTLEKYYPRGGAKAE
Haemonchus contortus serum-glucocorticoid-inducible kinase (SGK-1)
protein sequence (SEQ ID NO: 12)
MRKPPMVNCDVIVSVEKKPIFTIRIDSGHPVERRMRDFEKMYLKIAALPKKKFLQAEAKLQEKRRQ
WIVAFTQSLVANHYDNEEVRDFYALGDQQEKDESHVDLGPKEITTACPADFDFLTTLGKGSFGRV
FQVMHKESGKIYAMKVLSKEHIRRKNEVKHVMAELSVLKANFRHPFLVSLHFSFQNKEKLYFVLD
HLNGGELFTHLQKEKHFSEPRTRFYSAQIASALGYLHENNIVYRDLKPENLLLDKHGYVVLTDFGL
CKEGMMPNSLTSTFCGTPEYLAPEIILKKPYNVAVDWWCLGSVMYEMLYGLPPFYSRDHNEMYN
RIVNETLKIKKSISTASTDIITGLLQKDRNKRMGSKKDFKELEEHEFFKPIDWEKLLRHEIKAPFIPH
IDNETDVRNIAEDFVKIKINPASLAPQNLASTHQDHDFVNFTYVQKHDTMTNGLHANVQA

The composition or vaccine composition of the invention optionally includes an adjuvant.

The term “adjuvant” as used herein refers to an agent used to enhance the immune response of the immunised host to the immunising composition.

Suitable adjuvants for the vaccination of farmed or wild ruminant animals include but are not limited to oil emulsions such as Freund's complete adjuvant, Freund's incomplete adjuvant, squalane or squalene; mineral gels such as aluminium hydroxide, aluminium phosphate, calcium phosphate, calcium phosphate and alum; surfactants such as hexadecylamine, lysolecithin and methoxyhexadecylglcerol; polyanions such as dextron sulphate and carbopol; peptides such as muramyl dipeptide and dimethylglycine; or other adjuvants including QuilA, lipopolysaccharide, montanide, lipovant, bacterial flagellin, adjuvant 65, imiquimod, gamma inulin, guardiquimod, etc.

A preferred adjuvant is Quil A.

The composition or vaccine composition of the invention optionally includes a carrier.

The composition or vaccine composition of the invention may include a carrier selected from, but not limited to, solgel (a chitin based slow release compound), hollow mesoporous silicon nanoparticles (HMSNs), poly(d,l-lactide-co-glycolide) (PGC) nanoparticles, poly(d,l-lactic-coglycolic acid) (PGCA) nanoparticles, liposomes, virosomes, cochleate delivery vehicles, etc.

The composition or vaccine composition of the present invention can be given to an animal before any infection is detected to act as a preventative, or can be given as a treatment to infected animals.

The composition or vaccine composition of the present invention is preferably in a form for administering to an animal via subcutaneous or intramuscular injection.

The composition or vaccine composition of the present invention will be formulated for subcutaneous or intramuscular administration as a parenterally acceptable aqueous solution which is pyrogen-free and has a suitable pH, isotonicity and stability.

The composition or vaccine composition may contain salts, buffers, adjuvants or other substances which are desirable for improving the efficacy of the composition as would be understood by a skilled worker.

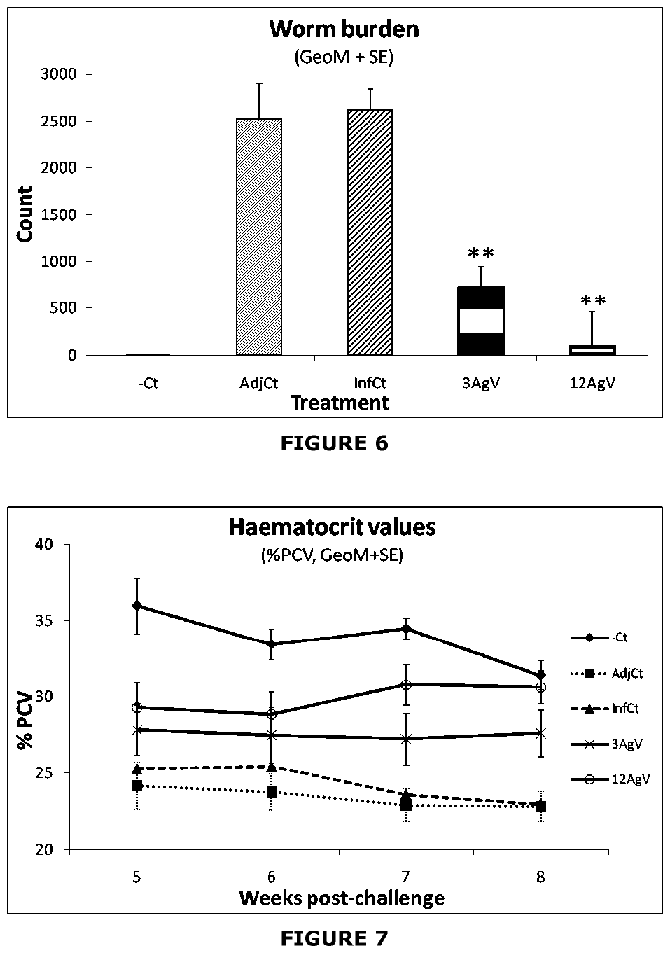

The composition or vaccine composition of the invention will be administered to an animal in a therapeutically effective amount, i.e. an amount that results in an immunologic response such as the production of desirable antibodies. As can be seen in the examples, sustained antibody responses to vaccines comprising three, eleven and twelve antigens has been demonstrated in sheep, calves and goats (see FIGS. 9, 10, 18 and 19). These antibody responses correlated with reduced worm burden in sheep and calves (see FIGS. 6 and 16) evidencing the efficacy of the vaccines. The goat trial was a proof of concept trial aimed at detecting an antibody response only. However, based on the results, it is expected that the vaccine would be efficacious in goats and other ruminants.

Typically, the amount of antigens administered to an animal is between about 30 μg-250 μg of each antigen, preferably between about 50 μg-200 μg, more preferably between about 75 μg-150 μg of each antigen, most preferably between 75 μg-100 μg of each antigen.

The composition or vaccine composition of the invention can be administered as a single or multiple dose of a therapeutically effective amount. Preferably, the composition or vaccine composition is administered twice, with a primary immunisation given followed by a booster 2-8 weeks later, preferably 3-4 weeks later. In some ruminants, such as young goats, a second booster may be required around 4-8 weeks after the first booster depending on antibody levels.

Additional doses can be administered as required to treat or prevent infection as would be understood by a skilled worker.

The composition or vaccine composition of the invention can be administered with anthelmintic agents such as levamisole, morantel, oxfendozole, monepantel and/or ivermectin to increase the overall FEC reduction rates and worm burden of the treated animals at the time of vaccination.

The composition or vaccine composition of the invention can also be administered with other vaccine treatments commonly administered to ruminants such as clostridial diseases (including pulpy kidney, tetanus, malignant oedema, black disease and black leg); bovine viral diarrhoea (BVD); footrot; leptospirosis; salmonella; scabby mouth, etc.

The composition or vaccine composition of the present invention is formulated to treat or prevent nematode worm infection in farmed or wild ruminants, particularly in sheep, cattle, goats, deer, buffalo, bison, camelids and llamas. Preferably the composition or vaccine composition of the invention is formulated to treat or prevent nematode worm infection in farmed animals including cattle, sheep, goats and deer, especially in young animals, less than one year old. In one aspect, the animal may be less than 6 months old. In another aspect, the animal is at least 3 months old.

The term “comprising” as used in this specification and claims means “consisting at least in part of”. When interpreting statements in this specification, and claims which include the term “comprising”, it is to be understood that other features that are additional to the features prefaced by this term in each statement or claim may also be present. Related terms such as “comprise” and “comprised” are to be interpreted in similar manner.

This invention may also be used to broadly consist in the parts, elements and features referred to or indicated in the specification of the application, individually or collectively, and any or all combinations of any two or more said parts, elements or features, and where specific integers are mentioned herein which have been equivalents in the art to which this invention relates, such known elements are deemed to be incorporated herein as if individually set forth.

The invention consists in the foregoing and also envisages constructions of which the following given examples only.

### Examples

### Trial 1

Immunisation of sheep with a vaccine composition comprising recombinant H. contortus enolase (EN), arginine kinase (AK) and ornithine decarboxylase (ODC).

### Materials and Methods

### Preparation of Antigen

Recombinant AK, EN and ODC were purified as described (Han et al., 2012; Umair et al 2013 a, b). Proteins were individually identified on gels stained with Coomassie Blue to confirm size and solubility.

### Recognition of Vaccine Antigens by Immune Lambs

Prior to the start of the trial, mucosal and systemic antibody responses against recombinant AK, EN and ODC were evaluated by performing enzyme-linked immunosorbent assay (ELISA) against immune or naive sheep saliva and serum using standard procedures.

### Animals

Use of experimental animals was approved by the AgResearch Animal Ethics Committee. 35 male Romney cross lambs (2-3 months old) were purchased from Ballantrae Farm, AgResearch Ltd and were transported to Flock house, drenched, weighed and tagged. Animals were randomly divided into five groups with seven animals in each group. The average group weight was the same in all groups before the start of the trial. All animals were suppressively drenched prior to the trial. Animals were grazed out-doors on paddocks with a history of no sheep grazing for at least last 3 years. Animals were divided into following groups:
Group 1: No treatment
Group 2: Alum control group (alum only)
Group 3: Vaccine in alum
Group 4: Quil A control group (Quil A in sol gel)
Group 5: Vaccine in Quil A and sol gel

### Vaccination Trial

Each animal in the vaccinated groups received 150 μg of each antigen SC and 500 μg orally in 1 mg/ml adjuvant. A final volume of 2.5 ml was given orally and 1 ml given SC. Groups 2 and 3 received vaccine or adjuvant only 3 times with 2 weeks interval whereas, animals of groups 4 and 5 were vaccinated only 2 times with 2 weeks interval. One animal from group 5 died soon after the trial commenced because of a clostridial infection. All other animals were then vaccinated with covexin 10 (anti-clostridial vaccine, MSD Animal Health) and no other animal died afterwards. One week after the last vaccination of the Alum groups, all animals except group 1 were infected with 2500 L3 H. contortus 2 times with one day interval. Serum and saliva samples were collected on a weekly basis. Animals were weighed and faecal samples were collected fortnightly prior to larval challenge and 3 times per week from day 16 post infection. All animals were killed 4 weeks after the larval challenge and abomasa collected for the adult worm recovery.

### Immune Assays and Parasitology

Antibody levels in serum and saliva were measured by ELISA. Eggs per gram faeces (EPG) were counted using the modified McMaster method in which each egg counted represented 50 eggs per gram faeces (Lyndal-Murphy, 1993). Adult worms were recovered from the abomasa in a volume of 7 litres and 10% was used to measure worm counts, male to female ratio and worm lengths. Worm lengths were performed using ImageJ software.

4 g faeces with known eggs output per gram from individual animals were collected at slaughter and cultured for larval development as described (Umair et al., 2013c). Briefly, faeces were cultured at room temperature and kept moist for 10 days and L3 developed from eggs were baermannised in water, collected and counted.

### Functional Activities of Recombinant Enzymes

Purified recombinant AK, EN or ODC were incubated in immune serum from animals of Quil A vaccine group at 25° C. for an hour and enzyme assays were performed to determine if antibodies in serum can inhibit the function of these enzymes. Recombinant enzymes were also incubated in naive serum to serve as controls. AK, EN and ODC assays were performed according to the protocol described (Umair et al., 2013a; Han et al., 2013; Umair et al., 2013b).

### Results and Discussion

Antibodies in saliva and serum from naturally immune sheep strongly reacted with the 3 recombinant proteins which demonstrated that the immune host is able to recognise these antigens.

### Antibody Responses

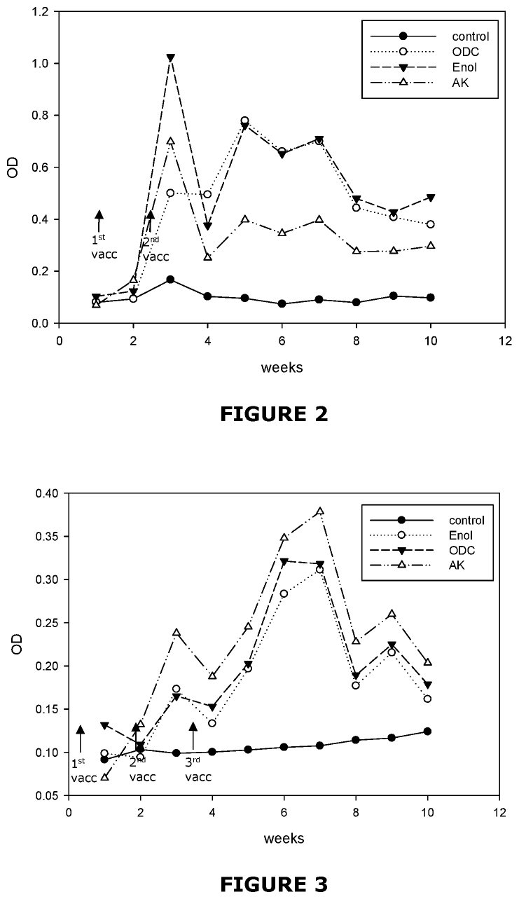

The serum IgG antibody response against recombinant AK, EN and ODC was measured by ELISA. Enzyme specific IgG levels were significantly higher in Quil A and alum vaccine groups compared to their respective control groups (FIGS. 2 and 3). The Quil A vaccine group had a much higher and faster developing antibody response compared to the alum group. FIG. 2 shows antibody responses in the Quil A groups with serum dilutions of 1:20,000. Serum samples from the alum group were tested at a dilution of 1:4000 (FIG. 3).

### Parasitology

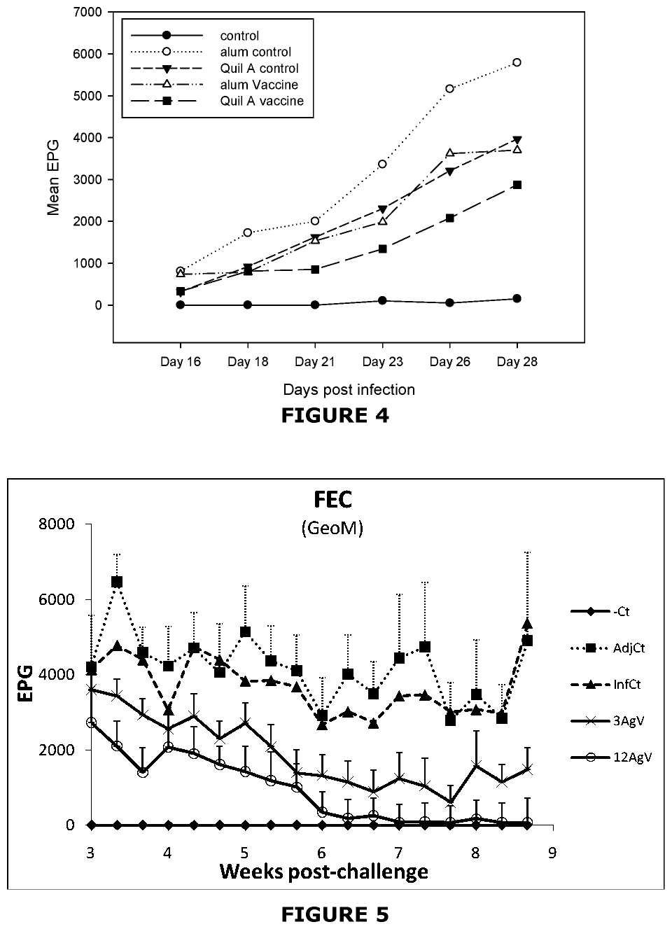

Mean EPG faeces among various groups are shown in FIG. 4. Mean egg counts were about 30% less in vaccinated groups than in their respective controls. It was interesting to note that mean egg counts were smaller in the control Quil A group than in the control alum group. It is possible that sol gel carrier mixed in Quil A might have some anti-parasitic activity on its own.

4 g faeces were cultured and the recovered larvae were counted. Proportions of larvae from the vaccine groups were not different compared to their respective control groups (data not shown), but the average length of female worms in the Quil A vaccine group was 18 mm compared to 19.6 mm in the alum vaccine group (data not shown).

When AK, EN or ODC assays were performed after incubation of enzymes with immune (Quil A vaccine group) or naive sera samples, a 25-30% reduction in the activity of all three enzymes was observed (Table 3 below). These results indicate that the antibodies from the sera of immunised lambs reduced the enzyme function.

The total number of worms and male: female ratio was also measured and compared between groups. The Quil A vaccine group showed the greatest reduction in worm number and was approaching significance (P=0.07). One animal of Quil A vaccine group died which made it difficult to analyse the results.

TABLE 3
AK, EN and ODC activities (nmoles min⁻¹ mg⁻¹ protein) (n = 2,
mean ± SD) in Quil A vaccine and control groups with incubation.
Enzymes were incubated in immune and naive sera prior to the assay.

| | Enzyme activity (Quil | Enzyme activity |
|---|---|---|
| | A control group | (Quil A vaccine group mean) |
| | mean) (incubated | (incubated in immune |
| Enzyme | in naive serum) | serum) |
| AK | 185 ± 10 | 150 ± 12 |
| EN | 580 ± 25 | 470 ± 20 |
| ODC | 2 ± 0.15 | 1.5 ± 0.10 |

### Conclusion

The results of this trial were very promising and show for the first time that recombinant antigens can be made that elicit significant immune responses when injected into sheep.

Further experiments with larger group size would be useful to conclusively establish if these antigens are protective and suitable vaccine targets.

### Trial 2

Immunisation of sheep with a vaccine comprising 3 or 12 H. contortus recombinant antigens.

### Materials and Methods

### Animals and Experimental Design

Use of experimental animals had been approved by the AgResearch Animal Ethics Committee. 42 newly-weaned cryptorchid male Romney cross lambs (2-3 months old) were purchased from Aorangi Farm (AgResearch Ltd), transported to Flock House, drenched, weighed and tagged. Animals were randomly divided into four groups (AdjCt, InfCt, 3AgV, 12AgV) with nine animals in each group. In addition, six animals were used in a non-infected control group (−Ct). The average group weight was identical in all groups before the start of the trial. Animals were fed on pasture with a history of no sheep grazing for at least last three years. These paddocks were grazed by cattle prior to and during the experiment.

The experiment was designed to test if vaccination with a combination of three nematode antigens (AK, EN, ODC) is effective in reducing the egg output and worm burdens; and to test whether or not the inclusion of additional antigens would have an additive effect and improve efficacy. Animals were divided into following groups:

TABLE 4
Treatment groups.

| | Number | |
|---|---|---|
| Group | (n) | Treatment |
| -Ct | 6 | No treatment, no infection control |
| AdjCt | 9 | Adjuvant control group (Quil A in Solgel) |
| InfCt | 9 | Infection control group |
| 3AgV | 9 | 3-Antigen vaccine, in Quil A and Solgel |
| 12AgV | 9 | 12-Antigen vaccine, in Quil A and Solgel |

### Vaccine Antigens and Formulations

### Three Antigen Vaccine (3AgV)

(i) Arginine kinase (AK)
(ii) Enolase (EN)
(iii) Ornithine decarboxylase (ODC)

Recombinant AK, EN and ODC were purified as described before (Han, Xu et al. 2012; Umair, Knight et al. 2013 a, b). Proteins were individually identified on gels stained with Coomassie Blue and size and solubility confirmed.

### Twelve Antigen Vaccine (12AgV)

In this group, the vaccine comprised the three antigens used above, namely:
(i) AK
(ii) EN
(iii) ODC
   and 9 others previously identified in a genome wide H. contortus RNAi screen (detailed in Appendix 1):
(iv) seryl tRNA synthetase (SRS-2)
(v) macrophage migration inhibitory factor 2 (MIF-2)
(vi) fatty acid synthetase (FASN-1)
(vii) NAD (P) H-dependant oxidoreductase (F36A2-3)
(viii) glutamyl tRNA synthetase (ERS-2)
(ix) aspartyl tRNA synthetase (DRS-1)
(x) transcriptional co-activator (CBP-1)
(xi) vacuolar ATPase (VHA-12)
(xii) serum-glucocorticoid-inducible kinase (SGK-1)

Recombinant proteins were expressed in E. coli (see Appendix 2). 150 μg of each antigen was formulated in 1 mg/mL Quil A as adjuvant and Solgel carrier for slow release.

### Vaccination Trial

Animals in groups 3AgV and 12AgV received a subcutaneous dose of 75 μg of each antigen in a final volume of 2.5 mL Quil A and Solgel. Two vaccinations were performed on Day 0 and Day 27. AdjCt animals received adjuvant only at the same times. Animals in AdjCt, InfCt, 3AgV and 12AgV were challenged with a trickle infection totaling 8000 H. contortus larvae given orally in three doses on Day 46, 47 and 48. −Ct animals served as non-infected controls to establish potential pasture contamination.

By Day 20 the weekly facial eczema spore count monitoring (Southern Rangitikei Veterinary Services) revealed spore counts of >15,000 which is above the trigger level of 12,000 for spore damage. Consequently, all animals were treated with a zinc bolus.

By Day 42 several lambs developed diarrhoea which was found to be due to low levels of infections with the cattle parasite Cooperia oncophora. Consequently, all animals were treated with a combination of Levamisol and Oxfendazole to remove any existing worm burden.

A drought condition developed which worsened during the course of the trial. This resulted in suboptimal nutrition of lambs. Three animals developed fly strike symptoms around Day 60 and were treated with Maggo. Consequently, all animals were treated with ZAPP to prevent future fly strike.

Serum samples were collected every two weeks for measuring antibody responses. Live weights were monitored monthly before and weekly after challenge. Packed cell volumes (PCV) were also monitored from week 5 to 8 after challenge. Faecal samples were collected fortnightly prior to larval challenge and three times per week from week 3 post infection. All animals were killed eight weeks after the larval challenge and abomasa collected for the adult worm recovery.

### Immune Assays

Serum antibody responses to vaccination were measured two weeks after the second vaccination by ELISA. A specific protocol was developed for each of the 12 vaccine antigens. Briefly, plates were coated with recombinant proteins, blocked and then incubated with a serial dilution of serum. Colour was developed with TMB and data expressed as OD.

### Parasitology Assays

Eggs per gram faeces (EPG) were counted using the modified McMaster method in which each egg counted represented 50 eggs per gram faeces. Adult worms were recovered from the abomasa in a volume of 7 L and 10% was used to measure worm counts, male to female ratio and worm lengths. Worm lengths were performed using ImageJ software.

### Results

### Parasitology

FEC showed a trend for a decline over time from week 3 to 8 after challenge in both vaccinated groups. Cumulative FEC were significantly lower in both vaccinated groups (P=0.039 and P=0.016, respectively) compared to non-vaccinated control groups. The highest level of reduction was seen in 12AgV animals followed by 3AgV (FIG. 5).

Counting the resident abomasal worm burden at the time of slaughter demonstrated that both vaccine groups harboured a significantly lower worm burden than the controls (AdjCt and InfCt, P<0.01). Worm burdens in 12AgV was lower than in 3AgV animals although this did not achieve statistical significant (P=0.1; FIG. 6).

### Haematocrit

Measuring haematocrit levels 5 to 8 weeks after challenge with H. contortus larvae demonstrated that infection with the parasite resulted in lower levels of PCV. Control animals already had low PCV values five weeks after challenge, which then declined further during the infection. The decline in PCV values in 3AgV and 12AgV was smaller compared to AdjCt and InfCt. At the end of the experiment values for 12AgV animals were similar to non-infected controls and values for both vaccine groups were significantly higher than for controls (FIG. 7). This demonstrates that the vaccine prevented the severe blood loss caused by H. contortus infection.

### Live Weight

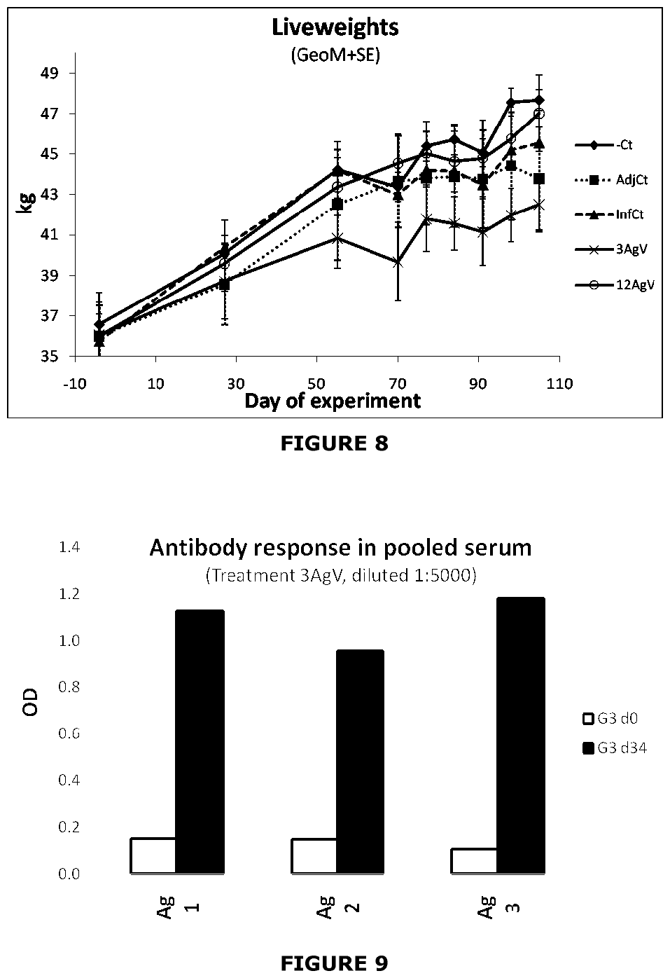

Live weights between the non-infected control group (−Ct) and 12AgV animals were very similar and did not differ significantly during the course of the experiment (FIG. 8). This demonstrates that vaccination with a combination of 12 antigens resulted in weight gains similar to non-infected control animals. Live weights for 3AgV animals were significantly lower at Wk 8 than for non-infected controls (P=0.024).

### Antibody Responses

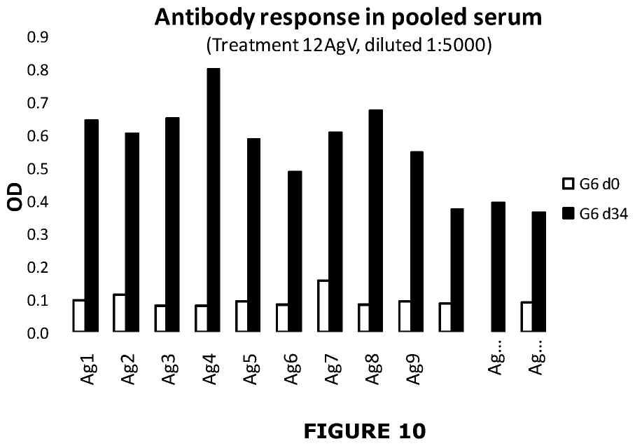

The serum IgG antibody response against 12 recombinant proteins was measured by ELISA. Enzyme-specific IgG levels significantly increased from pre-vaccination levels to those observed at Day 34 post-vaccination (FIG. 9 and FIG. 10). This demonstrates that two vaccinations induced a high serum antibody response to each individual antigen that was included in the respective vaccine.

### DISCUSSION

This study demonstrates efficacy of a recombinant vaccine against a nematode parasite of ruminants. Vaccination with a combination of recombinant parasite antigens resulted in a highly significant reduction in egg counts and worm burdens in young lambs that were challenged in the field with H. contortus. This reduction in worm burdens correlates with the control of Haemonchus induced blood loss.

### Conclusion

The present vaccine comprising multiple recombinant antigens resulted in a prototype vaccine that showed efficacy in young lambs under stringent field conditions.

### Trial 3

Immunisation of sheep with a vaccine comprising 3, 7 or 11 H. contortus recombinant antigens.

### Materials and Methods

### Animals and Experimental Design

Use of experimental animals had been approved by the AgResearch Animal Ethics Committee. 70 lambs, ˜3 month old cryptorchid, born at AgResearch Aorangi farm, were chosen for the study. Lambs were weaned and drenched at 10 weeks of age, as per farm practice. Lambs were given lucerne pellets for at least 1 week before they were transported to Grasslands animal facility. Lambs were transported to the indoor facility 2 weeks before the start of the trial and fed grass for 1 week and Lucerne pellets, Lucerne chaff, and FiberMix the following week. 55 lambs were selected based on the previous exposure to parasites (using CarLA saliva test) before the start of the trial and the remaining 15 lambs were returned to Aorangi.

The experiment was designed to test if vaccination with a combination of three nematode antigens (AK, EN, ODC) is effective in reducing the egg output and worm burdens; and to test whether or not the inclusion of additional antigens would have an additive effect and improve efficacy. Animals were divided into following groups:

TABLE 5
Treatment groups

| | Number | |
|---|---|---|
| Group | (n) | Treatment |
| NegCt | 5 | No treatment, no infection control (negative control) |
| PosCt | 10 | Infection control group (positive control) |
| AdjCt | 10 | Adjuvant control group (Quil A in Solgel) |
| 3AgV | 10 | 3-Antigen vaccine, in Quil A and Solgel |
| 7AgV | 10 | 7-Antigen vaccine, in Quil A and Solgel |
| 11AgV | 10 | 11-Antigen vaccine, in Quil A and Solgel |

### Vaccine Antigens and Formulations

### Three Antigen Vaccine (3AgV)

(i) Arginine kinase (AK)
(ii) Enolase (EN)
(iii) Ornithine decarboxylase (ODC)

Recombinant AK, EN and ODC were purified as described before (Han, Xu et al. 2012; Umair, Knight et al. 2013 a, b). Proteins were individually identified on gels stained with Coomassie Blue and size and solubility confirmed.

### Seven Antigen Vaccine (7AgV)

In this group, the vaccine comprised the three antigens used above, namely:
(i) AK
(ii) EN
(iii) ODC
   and 4 others previously identified in a genome wide H. contortus RNAi screen (detailed in Appendix 1):
(iv) seryl tRNA synthetase (SRS-2)
(v) macrophage migration inhibitory factor 2 (MIF-2)
(vi) fatty acid synthetase (FASN-1)
(vii) NAD (P) H-dependant oxidoreductase (F36A2-3).

Eleven antigen vaccine (11AgV)

In this group, the vaccine comprised the three antigens used above, namely:
(i) AK
(ii) EN
(iii) ODC
   and 8 others previously identified in a genome wide H. contortus RNAi screen (detailed in Appendix 1):
(iv) seryl tRNA synthetase (SRS-2)
(v) macrophage migration inhibitory factor 2 (MIF-2)
(vi) fatty acid synthetase (FASN-1)
(vii) NAD (P) H-dependant oxidoreductase (F36A2-3)
(viii) glutamyl tRNA synthetase (ERS-2)
(ix) aspartyl tRNA synthetase (DRS-1)
(x) transcriptional co-activator (CBP-1),
(xi) vacuolar ATPase (VHA-12)

Recombinant proteins were expressed in E. coli (see Appendix 2). 150 μg of each antigen was formulated in 1 mg/mL Quil A as adjuvant and Solgel carrier for slow release.

We note that the 11AgV in priority application NZ 780917 erroneously included SGK-1 as antigen (x) in the vaccine instead of CBP-1, as listed above.

### Vaccination Trial

Animals in groups 3AgV, 7AgV and 11AgV received two subcutaneous doses of 75 μg of each antigen in a final volume of 2.5 mL Adjuvant (Quil A formulated in Solgel, a chitosan based slow release) at 4 week interval. Adjuvant control group received only the adjuvant whereas the positive control group did not receive any treatment. Solgel is liquid at cool temperatures and forms a gel once inside the body which helps in the slow release of the vaccine.

Two weeks after the second vaccination, each animal, except the negative control group, was orally dosed with around 2,000 L3 Haemonchus contortus larvae daily for three days. A total of 6,000 L3 H. contortus larvae were given. All animals were weighed and bled weekly throughout the course of the trial, saliva sampled once every 4 weeks, faecal sampled monthly before the parasite challenge and twice weekly from day 16 post-infection. Weekly packed cell volume test was carried out for each animal to monitor the blood loss as the result of the infection, also the eye conjuctiva was checked weekly for anaemia. All animals were killed 10 weeks post second vaccination and the abomasa collected for parasitology.

Blood samples were processed and ELISA performed to compare the antibody levels between the vaccine and the control groups.

### Results

### Parasitology

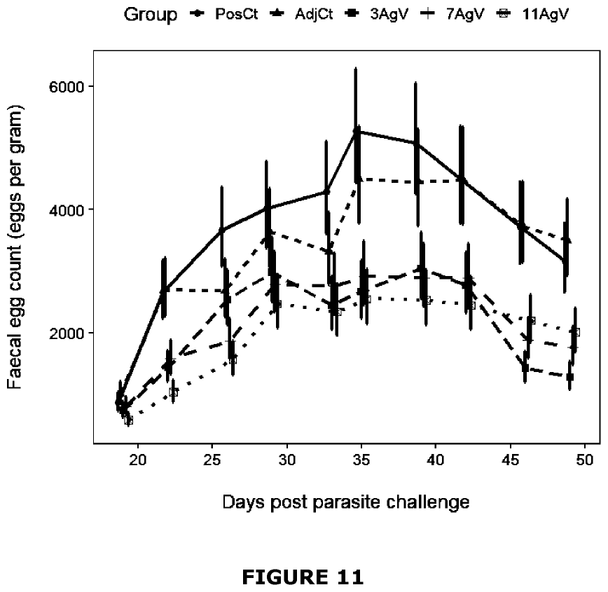

The faecal egg output in all the treatment groups from day 18 to day 48 post-infection is shown in FIG. 11. The egg output of animals of all three vaccine groups was significantly less than either of the positive control groups. 11AgV group had significantly less egg output at every sampling time point whereas the 3AgV and 11AgV groups had significant reduction at 9 sampling time points. Animals of the negative control group (not shown in FIG. 11) did not get the parasite challenge, hence, did not have any faecal egg output.

### Adult Worm Counts

The most surprising result of this trial was the reduction in the adult worm number in the treatment groups compared to the control groups as shown in FIG. 12. Total adult worm as well as total male and female worms were significantly reduced as a result of vaccination in all vaccine groups, 3AgV, 7AgV and 11AgV as compared to the control groups. 11AgV group showed the greatest reduction in the adult worms compared with control groups and the overall adult worm reduction was just over 60%. Adjuvant only (AdjCt) had no effect on the adult worm burdens and no differences between PosCt and AdjCt were recorded. There was no statistical difference between the treatment groups.

### Weight Gains

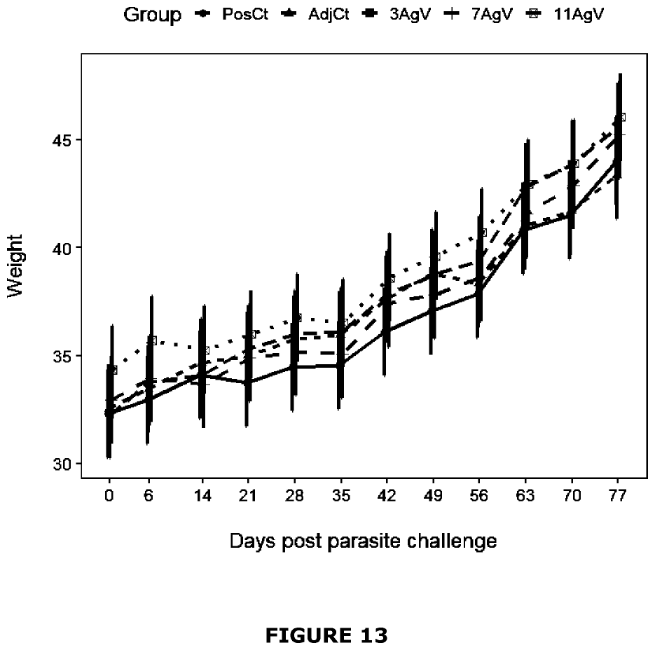

Vaccinated animals had on average higher weight gains compared to that of the non-vaccinated animals. The 11AgV group had on average 3 kg higher weight gain compared to AdjCt group, it was, however, statistically not significant (FIG. 13).

### Packed Cell Volume (PCV)

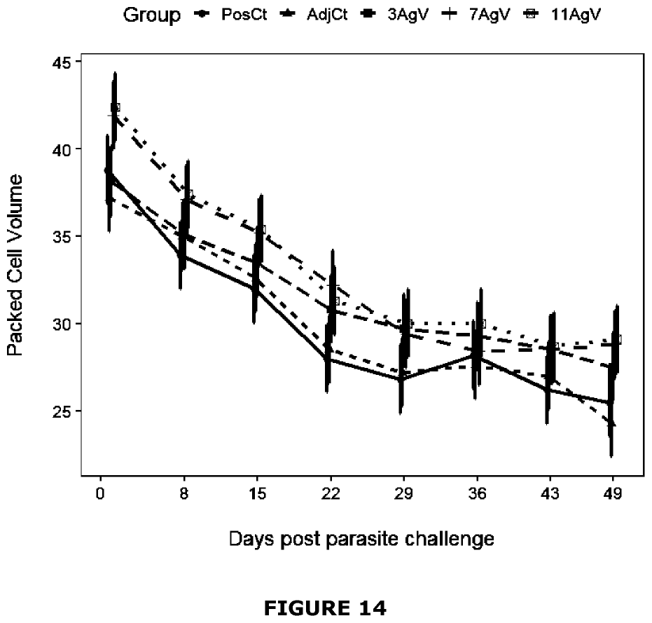

Animals of 7AgV and 11AgV lost significantly less blood to the Haemonchus infection compared to the control groups (FIG. 14). Although not statistically significant the 3AgV animal were less anaemic compared to the control groups. There was no difference between the two positive control groups. All the treatment animals were healthier and none had developed anaemia whereas 3 of the control groups animals were kept under observation because the haematocrit levels were very low.

### Antibody Response

Vaccination induced significantly higher serum antibody response in all the treatment groups compared to that of PosCt and AdjCt. Serum samples collected on weekly basis through the trial were tested against all 11 antigens and serum antibody responses to vaccination were measured in individual samples. The serum samples were diluted 1:2000, 1:8000 and 1:32000 and significantly higher antibodies were measured in all the treatment animals at all three dilutions. Saliva samples from individual animals were collected prior to the start of the trial and assayed for antibodies to the CarLA to access the level of pre-existing exposure to parasite infections. The CarLA levels in most of the animals were low to medium. The animals were divided into various control and treatment groups based on the weight and the CarLA level.

### Discussion

This study demonstrates efficacy of a recombinant vaccine against a nematode parasite of ruminants. Vaccination with a combination of recombinant parasite antigens resulted in a highly significant reduction in egg counts and worm burdens in young lambs that were challenged in the field with H. contortus. This reduction in worm burdens correlates with the control of Haemonchus induced blood loss. The results showed efficacy of all three vaccines comprising the core three antigens only (3AgV) as well as the 7AgV and 11AgV vaccines. The 11AgV group of animals had significantly fewer eggs in their faeces compared to controls. The vaccinated animals also had higher weight gains compared to that of the controls.

### Conclusion

The present vaccine comprising the three antigen vaccine (3AgV) with or without additional multiple recombinant antigens resulted in a prototype vaccine that showed efficacy in young lambs under stringent field conditions.

### Trial 4

Immunisation of calves with a vaccine comprising 3 or 11 H. contortus recombinant antigens.

### Materials and Methods

### Animals and Experimental Design

Use of experimental animals had been approved by the AgResearch Animal Ethics Committee. 27 Jersey calves, approximately 3 months old, were brought to Aorangi farm two weeks before the trial. Animals were drenched with a combination oral anthelmintic to remove any existing worm burden, weighed and saliva sampled. Animals were fed on previously prepared parasite-free pasture throughout the trial.

The experiment was designed to test if vaccination with a combination of three nematode antigens (AK, EN, ODC) is effective in reducing the egg output and worm burdens; and to test whether or not the inclusion of additional antigens would have an additive effect and improve efficacy. The animals were divided into the following groups:

| | Number | |
|---|---|---|
| Group | (n) | Treatment |
| NegCt | 3 | No treatment, no infection control (negative control) |
| PosCt | 8 | Infection control group (positive control) |
| 3AgV | 8 | 3-Antigen vaccine, in Quil A and Solgel |
| 11AgV | 8 | 11-Antigen vaccine, in Quil A and Solgel |

### Vaccine Antigens and Formulations

### Three Antigen Vaccine (3AgV)

(i) Arginine kinase (AK)
(ii) Enolase (EN)
(iii) Ornithine decarboxylase (ODC)

Recombinant AK, EN and ODC were purified as described before (Han, Xu et al. 2012; Umair, Knight et al. 2013 a, b). Proteins were individually identified on gels stained with Coomassie Blue and size and solubility confirmed.

### 11 Antigen Vaccine (11AgV)

In this group, the vaccine comprised the three antigens used above, namely:
(i) AK
(ii) EN
(iii) ODC
   and 8 others previously identified in a genome wide H. contortus RNAi screen (detailed in Appendix 1):
(iv) seryl tRNA synthetase (SRS-2)
(v) macrophage migration inhibitory factor 2 (MIF-2)
(vi) fatty acid synthetase (FASN-1)
(vii) NAD (P) H-dependant oxidoreductase (F36A2-3)
(viii) glutamyl tRNA synthetase (ERS-2)
(ix) aspartyl tRNA synthetase (DRS-1)
(x) transcriptional co-activator (CBP-1)
(xi) vacuolar ATPase (VHA-12)

Recombinant proteins were expressed in E. coli (see Appendix 2). 100 μg of each antigen was formulated in 1 mg/mL Quil A as adjuvant and Solgel carrier for slow release. Sol-gel is liquid at cool temperatures and forms a gel once inside the body, acting as a depot which helps in the slow release of the vaccine.

### Vaccination Trial

NegCt consisted of three animals used as tracer calves to detect the level of pasture contamination. The other three groups consisted of 8 animals each, as set out in the table above. The vaccine groups consisted of either 3 or 11 antigens (100 μg of each antigen) with the adjuvant (Quil A formulated in Solgel, a slow-release). Sol-gel is liquid at cool temperatures and forms a gel once inside the body, acting as a depot which helps in the slow release of the vaccine. Each of the 3AgV and 11AgV animals was vaccinated twice at four weeks intervals. Two weeks after the second vaccination, each calf (except NegCt) was orally dosed with 18000 L3 H. contortus, given as equal doses over four consecutive days. All animals were weighed weekly, blood was collected for packed cell volume and antibody titres, eye conjunctiva was checked weekly for anaemia, and twice-weekly faecal egg counts were carried out. All animals (except NegCt) were killed eight weeks post-second vaccination, and abomasa were collected for adult worm counts. The three NegCt calves were sold back to the farm.

### Results

### Parasitology

The faecal egg output of animals of the vaccinated groups from day 21 to day 38 post-infection was significantly lower than either of the positive control groups (FIG. 15). The 11AgV group had significantly lower egg output at every sampling time point, whereas the 3AgV group was significantly reduced at most sampling time points. Animals of the negative control group (not shown in FIG. 15) did not get the parasite challenge, hence, had zero faecal egg output.

### Adult Worm Counts

Total adult worms and total male and female worms were significantly reduced in the vaccination group compared to the control group (FIG. 16). No adult worms were recovered in any of the 11AgV group animals and the adult worms were reduced by 100% whereas there was 75% total adult worm reduction observed in the 3AgV group compared with the control group (PosCt).

### Packed Cell Volume (PCV)

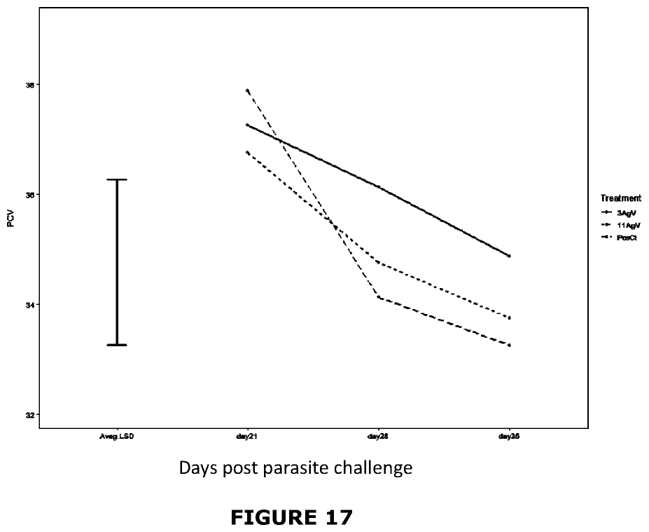

Vaccination also reduced the decline in haematocrit levels compared to the controls (FIG. 17). All the treatment animals were healthier, and none developed anaemia.

### Antibody Response

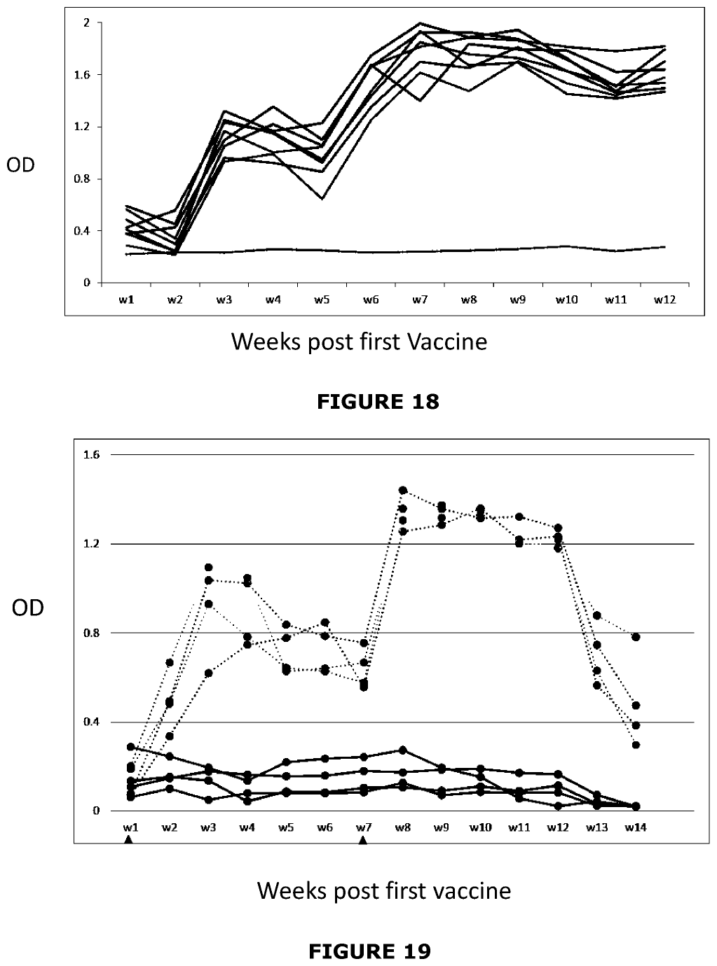

Serum samples collected weekly through the trial were tested against all 11 antigens, and serum antibody responses to vaccination were measured in individual samples. The serum samples were diluted at 1:1000, 1:4000 and 1:16000, and significantly higher antibodies were measured in all the treatment animals at all three dilutions. Saliva samples from individual animals were collected before the trial and assayed for antibodies to the CarLA to access the level of pre-existing exposure to parasite infections. The animals were divided into control and treatment groups based on weight and the CarLA level. The CarLA levels in most of the animals were very low. Vaccination induced significantly higher serum antibody response in both treatment groups than the control group (PosCt) (FIG. 18).

### Discussion

This study demonstrates efficacy of the 3 and 11 recombinant vaccine against a nematode parasite of ruminants. Vaccination with a combination of recombinant parasite antigens resulted in a highly significant reduction in egg counts and worm burdens in young calves that were challenged in the field with H. contortus. Vaccination resulted in a significant reduction in adult worm counts in both the 3Ag and the 11Ag groups. What is more surprising is that no adult worms were recovered from any of the 11Ag treated animals indicating 100% efficacy of the vaccine by day 38. Overall, vaccination positively impacted vaccinated calves, and the calves excreted fewer parasite eggs in their faeces. Also, vaccination resulted in significantly higher serum antibodies in all the vaccinated animals, and the antibody titres were maintained eight weeks after the second vaccination, suggesting protection against H. contortus infection could extend months after the vaccination. Data from the previous trials suggest that the recombinant Haemonchus vaccine works best when the parasite burden is low. H. contortus prefers sheep as a host and doesn't infect calves as well as sheep. Therefore, in this experiment, we passaged the parasites twice through calves before infecting the trial animals to increase the chances of infection. The calves were on moderate feed restrictions; even so, only 5-7% of the infection was established in the animals. As stated in the previous vaccine trials, the vaccine resulted in high antibody titres, yet there were no significant vaccine site reactions.

### Conclusion

The present vaccine comprising the three core antigens (3AgV) with or without additional multiple recombinant antigens resulted in a prototype vaccine that showed efficacy in young calves under stringent field conditions.

trial 5

Immunisation of goats with a vaccine comprising 11 H. contortus recombinant antigens.

### Materials and Methods

### Animals and Experimental Design

Use of experimental animals had been approved by the AgResearch Animal Ethics Committee. Eight male goat kids, ˜4 months old, were brought to the Grasslands Animal Facility two weeks before the trial. Animals were drenched, weighed, and grazed outdoors throughout the trial. This outdoor goat trail was designed to determine if the recombinant 11 antigen Haemonchus vaccine could induce serum antibodies in goats. The animals were divided into the following groups:

| | Number | |
|---|---|---|
| Group | (n) | Treatment |
| Control | 4 | No treatment, no infection control (negative control) |
| 11AgV | 4 | 11-Antigen vaccine, in Quil A and Solgel |

### Vaccine Antigens and Formulations

### 11 Antigen Vaccine (11Ag)

In this group, the vaccine comprised the following eleven antigens:
(i) AK
(ii) EN
(iii) ODC
(iv) seryl tRNA synthetase (SRS-2)
(v) macrophage migration inhibitory factor 2 (MIF-2)
(vi) fatty acid synthetase (FASN-1)
(vii) NAD (P) H-dependant oxidoreductase (F36A2-3)
(viii) glutamyl tRNA synthetase (ERS-2)
(ix) aspartyl tRNA synthetase (DRS-1)
(x) transcriptional co-activator (CBP-1)
(xi) vacuolar ATPase (VHA-12)

Recombinant proteins were expressed in E. coli (see Appendix 2). 75 μg of each antigen was formulated in 1 mg/mL Quil A as adjuvant and Solgel carrier for slow release.

### Vaccination Trial

The two groups consisted of 4 animals each, as set out in the table above. The vaccine groups consisted of 11 antigens (75 μg of each antigen) with the adjuvant (Quil A formulated in Solgel, a slow-release). Sol-gel is liquid at cool temperatures and forms a gel once inside the body, acting as a depot which helps in the slow release of the vaccine. Each of the 11AgV animals was vaccinated twice at seven weeks intervals. As this was only an antibody measurement trial, animals were not artificially infected with parasites. All animals were weighed fortnightly and weekly bleed for antibody titres. Serum samples were stored at −20° C. before antibody titres were measured by ELISA with the plates coated with all 11 antigens. All animals were killed ten weeks post-second vaccination and deeply buried as there was no tissue collection at slaughter.

### Results

Serum collected from the vaccinated kids showed significantly higher antibody titres compared with that from unvaccinated animals. The antibody titres seem to drop 3-4 weeks post-second vaccination (FIG. 19).

### Discussion

This trial was a proof of concept that vaccination results in the generation of increased vaccine-specific antibody levels in young goats. Interestingly, the antibody levels declined quite quickly, and all the kids lost most of their antibodies within six weeks after the second vaccination. Without being bound by theory, this is likely due to the fact that goats don't develop a distinct immunity against GIN infections or because goats don't develop a significant immune response to worms until they are older than 12 months. Therefore, young goats may benefit from a vaccine booster dose 4-8 weeks after the second vaccination, or vaccination at an older age (12 months or older). Further work is needed to confirm this. However, this trial successfully showed that the present 11AgV raised antigen-specific antibodies in young goats, which indicates that the 11AgV will work as a successful vaccine against Haemonchus in goats.

### Overall Conclusion

The anthelmintic vaccine of the present invention comprising three core recombinant antigens (AK, EN, ODC) (3AgV), and up to eight additional recombinant antigens (7AgV; 11AgV; and 12AgV) has shown surprising efficacy in a number of young ruminant animals (sheep, calves and goats). This recombinant Haemonchus vaccine has repeatedly shown a significant reduction in egg output. in the vaccinated animals. Vaccinated animals had less blood loss compared with the control animals. The recombinant Haemonchus vaccine is effective against the young ruminants and significantly reduces the number of adult worms, especially females who are metabolically more active than males because of their size and the number of eggs they produce. Although the vaccine removes around 80% of the adult worms, the remaining parasites help the animal develop immunity against the parasite. Generally, an animal becomes immune to a parasite after repeated exposure to the infection. The recombinant Haemonchus vaccine helps the animal to increase immunity against the infection. Without wishing to be bound by theory, it is believed that a small number of remaining worms within an animal will assist in the process of acquired immunity. Additionally, small numbers of parasites can easily be managed through good feeding practices. This vaccine will play a significant role in reducing the worm numbers in the areas where anthelmintic resistance is common and problematic.

### Appendix 1

Nematode Parasite (Haemonchus contortus) Vaccine Antigen Discovery

### Overview of Antigen Selection

The rationale for this study was the discovery of proteins (genes) essential for the viability of the parasitic nematode Haemonchus contortus that could be utilised as antigens for vaccine development. The discovery was approached from two directions.
1. An RNAi screen of Caenorhabditis elegans aimed specifically at uncovering genes with acute lethal or developmental arrest phenotypes was performed. A C. elegans RNAi library was produced from fragmented C. elegans genomic DNA in the dual T7 vector pL4440. More than 16,000 individual clones were screened for RNAi phenotype by performing an RNAi feeding assay. Of the screened clones 111 produced an RNAi phenotype (hit). A hit was defined as death or developmental arrest of worms during development from 1st stage (L1) to adult while feeding on E. coli that expressed a single RNAi clone. The inserts of clones with an RNAi phenotype were sequenced, identified by BLAST against C. elegans genome sequence and functionally annotated. The construction method used for the RNAi clone library resulted in a large number of chimeric clones where the insert consisted of two or more unrelated gene fragments due to insert-to-insert ligation. Where a chimeric insert was found C. elegans RNAi phenotypes from the Nematode Information Resource (Wormbase http://www.wormbase.org) were examined to determine those with a lethal or developmental arrest phenotypes.
2. A bioinformatics approach was undertaken whereby Wormbase was scrutinised for kinases, proteases and in some instances proteins associated with hits from the C. elegans RNAi screen (for example mif-2 and vha-12) that had lethal or developmental delay C. elegans RNAi phenotypes.

After this initial target selection a bioinformatic analysis of C. elegans RNAi hits was carried out to determine whether there was a likely H. contortus orthologue, either as an EST or a fragment of genomic sequence. H. contortus gene fragments for RNAi were cloned using available sequence for primer design. H. contortus RNAi was carried out by in vitro transcription of the RNAi clone followed by electroporation of the dsRNA into freshly hatched H. contortus L1s. Following electroporation, the larvae were cultured to infective third stage larvae (L3) in association with E. coli. The RNAi phenotype was scored at day 6. Control cultures should reach mature L3 stage by day 6. Assays were carried out in duplicate (technical replication) with at least two separately prepared batches of larvae and RNAi transcripts (biological replication). Developmental arrest or lethality where taken as evidence that the target was essential. H. contortus RNAi experiments were confined to testing the requirement for the target gene in the L1-L3 stages (outside the host). Expression of targets in adult parasitic worms was tested by RT-PCR using cDNA prepared from adults isolated from infected sheep. No RNAi testing against parasites in vivo was attempted because no methods exist to carry out RNAi against worms in sheep. However, “acute” RNAi phenotypes against fourth stage (L4) and adult C. elegans was carried out for some targets as a surrogate method by which a requirement for the target in adult worms could be assessed. A summary of C. elegans RNAi phenotypes, both reported in Wormbase and in some instances acute and H. contortus RNAi phenotypes are tabulated below (Table 5). Full length cDNA of selected genes were cloned, usually by 3′ and 5′ RACE, for expression in E. coli.

Targets that met the criteria above were selected for full-length cDNA cloning, usually by 3′ and 5′ RACE, for expression in E. coli. Full length cDNAs were cloned into E. coli expression vectors. The first choice where lac promoter vectors with N-terminal 6×His tags (Invitrogen). Where this failed, alternative vectors which utilise more stringent promoters (an arabinose promoter, and a lambda phage heat shock promoter) were used. His-tagged recombinant proteins were purified by Ni-affinity chromatography. Wherever possible, commercially available assays with known positive controls were used (this requirement was part of the target selection process). In some cases, no commercial kit or positive control was available.

TABLE 5

| | | | C. elegans | | | |
|---|---|---|---|---|---|---|
| | | | RNAi | C. elegans | H. contortus | |
| | | | Phenotype | acute | RNAi | H. contortus |
| | | Selection | (reported in | RNAi | Phenotype | RNAi |
| Target | Function | Method | Wormbase) | phenotype^{a} | (% alive)^{b} | Phenotype |
| srs-2 | seryl(S) tRNA synthetase | Ce RNAi screen | Emb | sterile adults | 49 (2) | yes |
| cbp-1 | transcriptional co-activator | Ce RNAi screen | Ste, Sck, Emb | nd | 53 (2) | yes |
| fasn-1 | fatty acid synthase | Ce RNAi screen | Emb, Lva | egg hatch defect | 59 (4) | yes |
| mif-2 | macrophage migration inhbitory factor 2 | Bioinformatics | | nd | 67 (2) | dev delay |
| F36A2.3 | NAD (P)H-dependant oxidoreductase | Ce RNAi screen | Unc, Egl | nd | 51 (3) | yes |
| ers-2 | glutamyl(E) tRNA synthetase | Ce RNAi screen | Emb, Lva | dev delay, egg | 43 (2) | yes |
| | | | | laying defect | | |
| drs-1 | aspartyl(D) tRNA synthetase | Ce RNAi screen | Emb, Ste, Lva | nd | 54 (2) | yes |
| sgk-1 | serum- and glucocorticoid-inducible kinase | Bioinformatics | Emb, Dev delay | nd | 64 (2) | yes |
| vha-12 | vacuolar ATPase, subunit B | Bioinformatics | Emb, Ste, Lva | nd | 69 (2) | yes |

^{a}nd is not done;
^{b}% alive is the number alive (total, L3, L2 or L1) as a percentage of the total number of alive and dead worms. Numbers bracketed are the number of independent experiments, 2 replicates/experiment.

### Appendix 2

Summary of Recombinant H. contortus Recombinant Protein Expression and Purification.

TABLE 6

| | | | | Activity Assay | Active |
|---|---|---|---|---|---|
| Milestone | Vector | Tag(s) | Purification | (NT = Not tested) | |
| Enolase (EN) | AY2.4 | Arabinose inducible, | Purification by Ni-NTA | | Y |
| | | C-terminal E-tag | agarose affinity | | |
| | | and 6xHis | chromatography. | | |
| Arginine | pET100 | | Ni-NTA agarose. | | Y |
| decarboxylase | | | | | |
| (AK) | | | | | |
| Ornithine | pET200 | N-terminal 6xHis | Ni-NTA agarose. | | Y |
| decarboxylase | | | | | |
| (ODC) | | | | | |
| srs-2 (seryl tRNA | AY2.4 | C-terminal E-tag | Ni-NTA agarose. | Aminoacylation of tRNA with | Y |
| synthetase) | | and 6xHis | | [U-¹⁴C]serine, precipitation with | |
| | | | | trichloroacetic acid (TCA) and | |
| | | | | precipitate captured on a glass | |
| | | | | fibre filter (adapted from methods | |
| | | | | of Taupin et al., 1997 and | |
| | | | | Weygand-Durasevic et al., 1993). | |
| ers-2 | pTrcHis | N-terminal 6xHis | Majority soluble, | Aminoacylation of total | N |
| (glutamyl tRNA | | | Ni-NTA | tRNA by [¹⁴C]glutamic acid. | |
| synthetase) | | | agarose. | | |
| drs-1 | pET200 | N-terminal 6xHis | 20° C. incubation, | Aminoacylation of total | N |
| (aspartyl tRNA | | | Ni-NTA | tRNA by [¹⁴C]aspartic acid. | |
| synthetase) | | | agarose. | | |
| cbp-1 | | | 15 ° C. incubation, | Fluorimetric assay based on | N |
| (transcriptional | | | purification by | that of Trievel et al., (2000). | |
| co-activator) | | | Glutathione | | |
| | | | sepharose affinity | | |
| | | | chromatography. | | |
| fasn-1 (fatty acid | pCL476 | | Ni-NTA agarose. | Cleave of esterase linkage in | Y |
| synthase) | | | | 4-methyllumbelliferone heptonate | |
| thioesterase | | | | (Richardson and Smith, 2007). | |
| domain | | | | | |
| mif-2 (macrophage | LAY2.4 | C-terminal E-tag | Soluble, Ni-NTA | Tautomerization of the coloured | Y |
| migration inhibitory | | and 6xHis | agarose. | dopachrome methyl ester | |
| factor) | | | | (Swope et al., 1998). | |
| f36A2-3 (NADPH- | pET200 | N-terminal 6xHis | Ni-NTA agarose. | Decrease in NADPH at 340 nm | N |
| dependant | | | | in the presence of potential | |
| oxidoreductase) | | | | substrates, including substrates | |
| | | | | for other oxidoreductase family | |
| | | | | members. | |
| sgk-1 (serum- | pTrcHis | N-terminal 6xHis | Ni-NTA agarose. | NT. | |
| glucocorticoid- | | N-terminal 6xHis | Ni-NTA agarose. | NT. | |
| inducible kinase) | | | | An assay for vacuolar ATPase | |
| vha-12 subunit B | | | | activity would require the | |
| (vacuolar ATPase) | | | | freconstitution of the two | |
| | | | | subunit proteins (Du and Grommet- | |
| | | | | Elhanan, 1999). | |

### REFERENCES

Lyndal-Murphy, M., 1993. Anthelmintic resistance in sheep. In: Corner, L. A., Bagust, T. J. (Eds.), Australian Standard Diagnostic Techniques for Animal Diseases. CSIRO, Melbourne, Australia, pp. 1-17.
Umair, S., Knight, J. S., Bland, R. J., Simpson, H. V., 2013a. Molecular and biochemical characterisation of arginine kinases in Haemonchus contortus and Teladorsagia circumcincta. Experimental Parasitology 134, 362-367.
Umair, S., Knight, J. S., Simpson, H. V., 2013b. Molecular and biochemical characterisation of ornithine decarboxylases in the sheep abomasal nematode parasites Teladorsagia circumcincta and Haemonchus contortus. Comparative Biochemistry and Physiology B 165, 119-124.
Umair, S., Ria, C., Knight, J. S., Bland, R. J., Simpson, H. V., 2013c. Sarcosine metabolism in Haemonchus contortus and Teladorsagia circumcincta. Experimental Parasitology 134, 1-6.
Han, K., L. Xu, et al. (2012). “Vaccination of goats with glyceraldehyde-3-phosphate dehydrogenase DNA vaccine induced partial protection against Haemonchus contortus.” Veterinary Immunology and Immunopathology 149 (3-4): 177-185.
Cho, Y, Jones, B. F, Vermeire, J. J, Leng, L, DiFedele, L, Harrison, L. M, Xiong, H, Kwong, Y-K. A, Chen, Y, Bucala, R, Lolis, E. and Cappello, M (2007) Structural and functional characterization of a secreted hookworm macrophage migration inhibitory factor (MIF) that interacts with the human MIF receptor CD74. J. Biol. Chem. 282:23447-23456
Du, Z and Grommet-Elhanan Z (1999) Refolding of recombinant a and B subunits of the Rhodospirillum rubrum F₀F₁ ATP synthase into functional monomers that reconstitute an active a1B1-dimer. Eur. J. Biochem 263:430-437
Hertweck M, Gobel C and Baumeister R (2004) C. elegans SGK-1 is the critical component in the Akt/PKB Kinase Complex to control stress response and life span. Dev. Cell 6:577-588
Marson, A. L, Tarr, D. E. K and Scott, A. L (2001) Macrophage migration inhibitory factor (mif) transcription is significantly elevated in Caenorhabditis elegans dauer larvae. Gene 278:53-62
Muramatsu H, Mihara H, Goto M, Miyahara I, Hirotsu K, Kurihara T. and Esaki N. (2005) A new family of NAD (P) H-dependent oxidoreductases distinct from conventional Rossman-fold proteins. J.Biosci. Bioeng. 99:541-547
Richardson R. D., Smith J. W. (2007) Novel antagonists of the thioesterase domain of human fatty acid synthase. Mol. Cancer Ther, 6:2129-2126
Swope M, Sun, H-W, Blake, P. R and Lolis E (1998) Direct link between cytokine activity and a catalytic site for macrophage migration inhibitory factor. EMBO J. 17:3534-3541
Taupin, C. M-J., Hartlein, M. and Leberman, R. (1997). Seryl-tRNA synthetase from the extreme halophile Haloarcula marismortui: Isolation, characterisation and sequencing of the gene and its expression in Escherichia coli. Eur. J. Biochem. 243:141-150
Weygand-Durasevic I., Ban, N., Jahn, D. and Soll, D. (1993). Yeast seryl-tRNA synthetase expressed in Escherichia coli recognises bacterial serine-specific tRNAs in vivo. Eur. J. Biochem. 214:869-877 Wormbase http://www.wormbase.org/
Zang X, Taylor P, Wang, J. M, Meyer, D. J, Scott, A. L, Walkinshaw, M. D and Maizels R. M (2002) Homologues of human macrophage migration inhibitory factor from a parasitic nematode J. Biol. Chem. 277:44261-44267
Trievel, R. C., Li, F-Y and Marmorstein R., 2000. Application of a fluorescent histone acetyl transferase assay to probe the substrate specificity of the human p300/CBP-associated factor. Anal. Biochem. 287, 319-328.
Victor, M., Bei, Y., Gay, F., Calvo, D., Mello, C. and Shi, Y., 2002. HAT activity is essential for CBP_1-dependent transcription and differentiation in Caenorhabditis elegans. EMBO Reports 3, 50-55.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

1. A composition or vaccine composition comprising the recombinant H. contortus antigens:
(i) enolase (EN);
(ii) arginine kinase (AK); and
(iii) ornithine decarboxylase (ODC),
or antigenic fragments thereof, together with a veterinary acceptable carrier or diluent.

2. The composition or vaccine composition of claim 1, further comprising one or more recombinant H. contortus antigens selected from the group consisting of:
(iv) seryl tRNA synthetase (SRS-2);
(v) macrophage migration inhibitory factor 2 (MIF-2);
(vi) fatty acid synthetase (FASN-1);
(vii) NAD (P) H-dependant oxidoreductase (F36A2-3);
(viii) glutamyl tRNA synthetase (ERS-2);
(ix) aspartyl tRNA synthetase (DRS-1);
(x) transcriptional co-activator (CBP-1);
(xi) vacuolar ATPase (VHA-12); and
(xii) serum-glucocorticoid-inducible kinase (SGK-1),
or antigenic fragments thereof.

3. The composition or vaccine composition of claim 2, comprising at least one, at least two, at least three, at least four, at least five, at least six, at least seven, at least eight, or at least nine, of the antigens (iv) to (xii).

4. A composition or vaccine composition comprising the H. contortus recombinant antigens:
(i) enolase (EN);
(ii) arginine kinase (AK);
(iii) ornithine decarboxylase (ODC);
(iv) seryl tRNA synthetase (SRS-2);
(v) macrophage migration inhibitory factor 2 (MIF-2);
(vi) fatty acid synthetase (FASN-1);
(vii) NAD (P) H-dependant oxidoreductase (F36A2-3);
(viii) glutamyl tRNA synthetase (ERS-2);
(ix) aspartyl tRNA synthetase (DRS-1);
(x) transcriptional co-activator (CBP-1);
(xi) vacuolar ATPase (VHA-12); and
(xii) serum-glucocorticoid-inducible kinase (SGK-1),
or antigenic fragments thereof, together with a veterinary acceptable carrier or diluent.

5. The composition or vaccine composition of any one of claims 1-4, further comprising an adjuvant.

6. The composition according to claim 5, wherein the adjuvant is selected from one or more of the group consisting of alum, Quil A, Freund's complete adjuvant, Freund's incomplete adjuvant, lipopolysacharride, monophosphoryl lipid A, montanide, lipovant, bacterial flagellin, adjuvant 65, gamma inulin, algammulin, imiquimod, guardiquimod and murimyl dipeptide.

7. The composition or vaccine composition of any one of claims 1-6, further comprising a carrier.

8. The composition of claim 7, wherein the carrier is selected from one or more of the group consisting of a chitin-based slow release compound (sol-gel), hollow mesoporous silicon nanoparticles (HMSNs), poly(d,l-lactide-co-glycolide) (PGC) nanoparticles, poly(d,l-lactic-coglycolic acid) (PGCA) nanoparticles, liposomes, virosomes and cochleate delivery vehicles.

9. A method of reducing parasitic nematode worm burden in a farmed or wild ruminant animal, said method comprising administering an effective amount of the composition or vaccine composition of any one of claims 1-8 to said ruminant animal on one or more occasions, whereby parasitic worm burden reduction is measured by a reduced faecal egg count (FEC), and/or an increase in expulsion of larvae and/or adult nematode worms.

10. A method of inducing an immune response in a farmed or wild ruminant animal to treat or protect said animal against infection by parasitic nematodes, said method comprising administering an effective amount of the composition or vaccine composition of any one of claims 1-8 to said animal on one or more occasions, wherein induction of an immune response is measured by the presence of protective antibodies against one or more specific antigens present in said composition or vaccine composition.

11. A method of stimulating or boosting acquired immunity in a farmed or wild ruminant animal to treat or protect said animal against infection by parasitic nematodes, said method comprising administering an effective amount of a composition or vaccine composition of any one of claims 1-8 to said animal on one or more occasions, wherein stimulation or a boost of said acquired immunity is measured by one or more of: the presence of protective antibodies against one or more specific antigens present in said composition or vaccine composition; an increased level of cytokines; a reduced FEC; and/or expulsion of larvae and/or adult nematodes.

12. A method of treating or preventing a nematode infection in a farmed or wild ruminant animal comprising administering an effective amount of a composition or vaccine composition of any one of claims 1-8 to said animal.

13. A use of the recombinant H. contortus antigens (i) enolase (EN), (ii) arginine kinase (AK), and (iii) ornithine decarboxylase (ODC), or antigenic fragments thereof, in the manufacture of a composition or vaccine composition for reducing nematode parasitic worm burden in a farmed or wild ruminant animal.

14. A use of the recombinant H. contortus antigens (i) enolase (EN), (ii) arginine kinase (AK), and (iii) ornithine decarboxylase (ODC), or antigenic fragments thereof in the manufacture of a composition or vaccine composition for stimulating or boosting acquired immunity in a farmed or wild ruminant animal to treat or protect said animal against infection by parasitic nematodes.

15. A use of the recombinant H. contortus antigens (i) enolase (EN), (ii) arginine kinase (AK), and (iii) ornithine decarboxylase (ODC), or antigenic fragments thereof, in the manufacture of a composition or vaccine composition for treating or preventing a nematode infection in a farmed or wild ruminant animal.

16. A use as claimed in any one of claims 13-15, wherein the composition or vaccine composition further comprises one or more of antigens (iv)-(xii), as defined in claim 2.

17. A method as claimed in any one of claims 9-12, or a use as claimed in any one of claims 13-16, wherein the farmed or wild ruminant animal is selected from the group consisting of sheep, cattle, goat, deer, buffalo, bison, camelids and llamas.

18. A method or use as claimed in claim 17, wherein the farmed or wild ruminant animal is a young animal, less than one year old.

19. A method or use as claimed in claim 18, wherein the farmed or wild ruminant animal is less than 6 months old.

20. A method as claimed in any one of claims 9-12, or a use as claimed in any one of claims 13-16, wherein the parasitic nematode worms are selected from one or more of the group consisting of Trichostrongylus colubriformis, Haemonchus contortus, Haemonchus placei, Ostertagia (Teladorsagia) circumcincta, Cooperia curticei, Nematodirus spathiger, Trichostrongylus axi, Trichostrongylus vitrinus, Ostertagia ostertagia, Cooperia oncophera, Nematodirus brasiliensis, Dictyocaulus eckerti, Strongylus vulgaris, Toxascaris vitolorum, Nematodirus filicollis, Ashworthius sidemi, Mecistocirrus digitatus, Bunostomum trigonocephalum, Trichuris discolour and Toxacara vitulorum.


SEQUENCE LISTING (12 sequences)

SEQ ID NO: 1 - protein, 433 aa, Haemonchus contortus
MPITKIHARQIYDSRGNPTVEVDLYTEKGVFRAAVPSGASTGVHEALELRDQDKKVHHGKGVLKAVANINDKIAPALIAKNFCVTQQRDIDQFMLALDGTENKSNLGANAILGVSLAVAKAGAVHKGMPLYKYIAELAGVSKVILPVPAFNVINGGSHAGNKLAMQEFMILPVGATSFHEAMRMGSEVYHHLKAEIKKRYGLDATAVGDEGGFAPNIQDNKEGLDLLKTAIDLAGYTGKISIGMDVAASEFYKQGKYDLDFKNPKSDPSKWLTGDQLAALYQTFIKEYPVVSIEDAFDQDDWDNWGKLKASTNIQLVGDDLTVTNPKRIRLAIDKKSCNCLLLKVNQIGSVTESIEAAKLSRSNGWGVMVSHRSGETEDTFIADLVVGLATGQIKTGAPCRSERLAKYNQLLRIEEELGKDAVYAGQNFRNPV

SEQ ID NO: 2 - protein, 359 aa, Haemonchus contortus
MSVPPEIIKKIEDGYQTLQNAKDCHSLLKKYLTKEVVDQLKDKKTKLGATLWDVIQSGVANLDSGVGVYAPDAEAYTLFKPLFDPLIQDYHNGFSPSQKQPATDLGEGKTAQLVDLDPEGKYINSTRVRCGRSLQGYPFNPCLTEANYLEMEAKVKKIFENISDPELQGTYYPLDGMTKEVQNQLIKDHFLFKEGDRFLQAANACRYWPKGRGIFHNKNKTFLVWANEEDHLRIISMQNGGNVGQVLERLIKGVKIIQAQAPFSRDDRLGWLTFCPSNLGTTVRASVHIRLPKISAKPDFKKICDDLKLQIRGIHGEHSDSEGGVYDISNKARLGLTEFEAVKQMYDGVKHLIELEKKA

SEQ ID NO: 3 - protein, 422 aa, Haemonchus contortus
MTMITQMELIGDSKVGIADGEVDALSMCRQIARNYDKDNIDEALCWSTDVVSTDSFVKRELPMIEPFYAVKCNTDRVLVRTLAALGTGFDCASREEIDIVMDLGVSAERIVYANPCKTRSFITHAKERNVSMMTFDSAEELAKVAQLHPQAKMILRIAVSDPTARCPLNLKFGADPVKMAPQLLVHAQELGVDVIGISFHVGSGCNDPTAYREALAHARHLIELGRGLGLDMTLVDLGGGYPGTPQQTSFEDIAAVIRSAVDEYLPPEFGVRLIAEPGRFFAAAPFTLVCNIIHATEVSAEKITKRPEDVEERGFMYYVNDGVYGSFNCILFDHVQPVGTPLFDEIAQEYPSTIWGPTCDSLDKIEDQKLMRMMSVGEWIVYRNMGAYTCSASTTFNGFQRPNAIYMINRKNWARISTSPNV

SEQ ID NO: 4 - protein, 486 aa, Haemonchus contortus
MVLDMDLFREEKGGNPEAIRNSQRQRYCDPSIVDKVIELDQAWRKERFLLDVLNRQKNVLSKAIGEKVKKKEAQGTDDNVDDSIVSKLESLKVEDLSALTVVQIKKLRVLLDEKMNETKASMEKLEDDRHQSLIQIGNIIHHSVPVSDDEANNRVERTHGDITSRKKYSHVDLVVMIDGFDGERGTAVAGGRGYFLKGPLVFLEQAIIQLALQKLGEKGFTPLYTPFFMRKEVMQEVAQLSQFDGELYKVNSKGSEVLGDNSIDEKYLIATSEQPIAAFHRNEWIKESDLPIKYAGISTCFRQEVGSHGRDTRGIFRVHQFEKVEQFVICSPLNNESWKIFDEMINNAEEYCQLLGIPYQIVCIVSGELNNAASKKLDLEAWFPGSGAFRELVSCSNCTDYQARRLKVRYGMTKKMDGEVPFVHMLNATMCATTRVLCALLENYQTEDGITVPEVLHPFMPAKYRTFIPFVKPAPIDEESKKKSGK

SEQ ID NO: 5 - protein, 118 aa, Haemonchus contortus
MPMVRVATNLPDKDVPANFEERLTDLLAESMNKPRARIAVEMMAGQRIMHGGVRNPVVLIKVESIGALDPDSTIRHTQKVTQLCTEVLHVPKDKVIISYFDLAPTNVGFAGTTVAAAT

SEQ ID NO: 6 - protein, 328 aa, Haemonchus contortus
TRALQPTELDMKKESERDAEQNTVEMLEKQMNQLFKMRVDVNDLDPQDIVVKCNKIEEGPVTFFVHSIEGIATPLKRVMTKCTFPVYCFQSTKEVPQDSIESVAKCYIREMKKIQPAGPYRIVGYSYGACIGFEMATMLQESDGPSAVERLILLDGSHLYMQTYRNVYRMAFGVTGDTLVNNPLFESEIMCAMTLRFANVDYKKFRVELLQQPGFKARIQKVVDTVMTTGLFKSPETIAFACEAMRSKFLMADKYKPERKFTGLITLVRAEQGAAREEDVGTDYGISQVADDSKVYVVEGDHDTFVQGKSSAKTVAIINELIKETYKC

SEQ ID NO: 7 - protein, 384 aa, Haemonchus contortus
MIIKKQFDESEEIVVSKKELRSFVLNCLEKVSCSPGHAQQLADILICSDYRGHYSHGLNRLHIYVNDLAEKSTLSDGEPLIIKQKGATAWVDGCNLLGPVVGNFCMKLAIQKARTHGIGWVVAKNSNHFGIAGWYAESALQQGLVGMAFTNTSPCVFPTNSAEKSLGSNPICLAAPAANGDSFFLDMASTTVAYGKIEVVDRRGGKRIPRAWGADADGIETQDPKEVLNGGGLQPLGGSEATGGYKGTGLCMMVEILCGIMAGSSFGKSIRKWQTTDESANLGQCFVAIDPECFAPGFGERLSCFLDETRDLKPVDPSYPVQVAGDPERAHMYMCDELDGIVYKKSQLEHLNKLANDLGVHMFKAKKMTSSLETDGQSRIELEV

SEQ ID NO: 8 - protein, 1016 aa, Haemonchus contortus
MVAVKQQLVINAKQKEVPYAAALAIAFGGYNPCLSIAFNEKEPVGMNMDGSLIRNDVAIARIVAQSLGLPEFTGSTCFEIAKIDEVLTLCEKLQEKFSALFNAIVEDQRFVAAHMMVGKFTTAKIAKPLSKEKQRDEGKFVELPGAEKGKVVVRFPPEASGYLHIGHAKAALLNQYYQQTFEGQLIMRFDDTNPAKENAHFEKVIKEDLAMLNIIPDRWTHSSDYFELMLQMCEKLLREGKAYVDDTDTETMRKEREERVESKNRSLSPEANLVLWEEMKKGTERGLQCCVRIKIDMQSNNGAMRDPTIYRCKPEEHVRTGMKYKVYPTYDFACPIVDSIEGVTHALRTTEYTDRDDQYYFICDALGLRKPFIWSYARLNMTNTVMSKRKLTWFVNEGLVEGWDDPRFPTVRGVMRRGMTVEGLRQFIIAQGGSRSVVMMEWDKIWSFNKKVIDPVAPRYTALETTAIVPVFISTPVVVQDAEVPLHPKNADVGKKTIWHSAKLLVEQVDAQEMKSGDTVTFVNWGNIKIVSVNKKNGTVSEIHAVLDLANQDYKKTMKVTWIAEADIPSAACIPVVAIEYDHIISKAVVGKEEDWKNFINYESVHYTKMLGEPALRSVRKGDIIQLQRKGFYICDHDYQPKSEFSGAESPLLLIYIPDGHVKEPVNKPKPSSVVAASTGKPGDALDLYKLIEAQGNTVRDLKSKDPKAESTKMAVQKLLELKKQYSEVTGQEYKPGKVPEPSNKVAASTTNESLALYMKIEAQGELVRTEKAKDAKSEASKAAIATLLELKKEYKEKTGQEYKAGQPPATTAPSVGTPPGAITEPSTIYAEIEAQGELVRKEKAKDPKSETAKAAIAKLLDLKKQYKEQTGQDYKPGQQATSLKSPSLGSGGDAISLYSEIEAQGNLVRQEKAKDAKSEAAKAAIAKLLELKKKYEEVTGHPYKPGQPPAETPSSPQKTAFDESALYEEIKAQGDLVRQEKQKDAKSDASKAAIQKLLDLKKLYKEKTGQEYKPK

SEQ ID NO: 9 - protein, 521 aa, Haemonchus contortus
MNDAAEGGEKKLSKKELNKLAKAAKIAELKAQKAASQPKEDEGEDVSVGMYGSYGMIQSADKKDIVFTKLNKIEPDLDGQEVWVRGRVHAIRSKGKTCFLVLRQQFYTAQVTLFVGEKISKQMLKFVSNISKESIVDIQGLVGKVDVQIESCTQKNAELHAIQVFVVSAAEPRLPLQIEDASRRADNTDGLAAVNLDTRLDNRVLDLRTTTTQGIFSLQAGVCKLFRDTLTERGFVEIHTPKIISAASEGGANVFTVSYFKGSAYLAQSPQLYKQMAIAGDFGKVFTIGGVFRAEDSNTHRHMTEFVGLDLEMAFNFHYHEVLETIGSVLISIFKGLKKDYAAEIEAVGRQYPAEPFEFCEPALVLKYPDAVKMLREDGVEMGDEDDLSTPVEKQLGRLVKEKYKTDFFILDKFPLAVRPFYTMPDPHDPRYSNSYDMFMRGEEILSGAQRIHDAEFLVERAKHHNIELEKIQAYIDSFKYGCPPHAGGGIGLERVTMLFLGLHNIRLASMFPRDPKRITP

SEQ ID NO: 10 - protein, 453 aa, Haemonchus contortus
ERYTYCLKCFDASPPEGISLSENPNDQSNMAPKDKFVQMKNNVIDYEPFEVCKYCHRKWHRICALYDKKVFPEGFICDTCRKEKNYPKPKNRFMAKRLPHNKLSQFLEDRVNTFLKKALSNSPEQYEVIIRTLCVQDKEVEVKPLMKSKYGPQGFPDRFNYRTKAVFAFEIIDGVEVCFFGLHVQEYGSNCKEPNARRVYIAYLDSVHFFQPRELRTDVYHEILLGYLDYVKRLGYTMAHIWACPPSEGDDYIFHCHPPEQKIPKPKRLQDWYKKMLEKGVTEKTVVEFKDIYKQARDDNLTTPMSLPYFEGDFWPNVIEDCIREAGNEEAQRRKEVAEADEEDDDIFQSGDNGKKKSSKNKKNNLKKNSKMNKKKQGNSTGNEVADKLYSQFEKHKEVFFTIRLVTQQSALSLPDIVDPDPLMASDMMDGRDTFLTRARDEHWEFSSLRRAK

SEQ ID NO: 11 - protein, 494 aa, Haemonchus contortus
MAAVDVNKGITSHKTATIRNYNTQPRLIYKTVTGVNGPLVILNDVKFPQFNEIVHITLPDGSKRSGQVLEITRNKAVVQVFEGTSGIDAKNTICEFTGDILRSPVSEDMLGRIFNGSGKPIDKGPPVLAEDFLDINGQPINPWSRIYPEEMIQTGISAIDVMNSIARGQKIPIFSAAGLPHNEIAAQIVRQGGLVQLPERKHDASDSNFAIVFAAMGVNMETARFFKQDFEENGSMENVCLFLNLANDPTIERIITPRLALTAAEFFAYQCEKHVLVVLTDMSSYAEALREVSAAREEVPGRRGFPGYMYTDLATIYERAGRVEGRDGSITQIPILTMPNDDITHPIPDLTGYITEGQIYVDRQLHNRQIYPPINVLPSLSRLMKSAIGEGMTREDHSDVSNQLYACYAIGKDVQAMKAVVGEEALSSDDLLYLEFLVKFEKNFITQGNYENRTVFESLDIGWQLLRIFPREMLKRIPESTLEKYYPRGGAKAE

SEQ ID NO: 12 - protein, 454 aa, Haemonchus contortus
MRKPPMVNCDVIVSVEKKPIFTIRIDSGHPVERRMRDFEKMYLKIAALPKKKFLQAEAKLQEKRRQWIVAFTQSLVANHYDNEEVRDFYALGDQQEKDESHVDLGPKEITTACPADFDFLTTLGKGSFGRVFQVMHKESGKIYAMKVLSKEHIRRKNEVKHVMAELSVLKANFRHPFLVSLHFSFQNKEKLYFVLDHLNGGELFTHLQKEKHFSEPRTRFYSAQIASALGYLHENNIVYRDLKPENLLLDKHGYVVLTDFGLCKEGMMPNSLTSTFCGTPEYLAPEIILKKPYNVAVDWWCLGSVMYEMLYGLPPFYSRDHNEMYNRIVNETLKIKKSISTASTDIITGLLQKDRNKRMGSKKDFKELEEHEFFKPIDWEKLLRHEIKAPFIPHIDNETDVRNIAEDFVKIKINPASLAPQNLASTHQDHDFVNFTYVQKHDTMTNGLHANVQA